(19) United States
(12) Patent Application Publication
(10) Pub. No.: US 2021/0121290 A1
(43) Pub. Date: Apr. 29, 2021

(54) SHEET MATERIAL FOR MEDICAL DEVICES

(71) Applicant: Abbott Laboratories, Abbott Park, IL (US)

(72) Inventors: Yousef F. Alkhatib, Edina, MN (US); Jay Reimer, Saint Paul, MN (US); Paul E. Ashworth, Danbury, WI (US); Keith T. High, White Bear Lake, MN (US)

(73) Assignee: Abbott Laboratories, Abbott Park, IL (US)

(21) Appl. No.: 16/899,084

(22) Filed: Jun. 11, 2020

Related U.S. Application Data
(60) Provisional application No. 62/925,410, filed on Oct. 24, 2019.
(60) Provisional application No. 62/925,418, filed on Oct. 24, 2019.

(51) Int. Cl. A61F 2/24

(52) U.S. Cl. CPC A61F 2/2418; A61F 2/2415; A61F 2002/0086; A61F 2210/0076; A61F 2/2433

(57) ABSTRACT

At least a portion of sheet materials for use in medical devices is formed from polymeric materials. The sheet materials may be partially coated or fully coated with one or more additional layers of a polymer. The sheet materials may be used for the leaflets and/or cuffs of prosthetic heart valves and as a component of other medical devices.

### CROSS-REFERENCE TO RELATED APPLICATIONS

The present application claims the benefit of the filing dates of U.S. Provisional Patent Application Nos. 62/925,410, filed Oct. 24, 2019; and 62/925,418, filed Oct. 24, 2019, the disclosures of all of which are hereby incorporated by reference herein.

### BACKGROUND OF THE DISCLOSURE

The present disclosure relates to synthetic sheet materials that can be used in various medical devices and the medical devices including the synthetic sheet materials. For purposes of discussing the state of the art, however, prosthetic heart valves, and particularly collapsible/expandable prosthetic heart valves useful for delivery through a catheter or trocar, will be exemplified.

Prosthetic heart valves, including surgical heart valves and expandable heart valves intended for transcatheter aortic valve replacement (“TAVR”) or transcatheter mitral valve replacement (“TMVR”), are well known in the patent literature. (See U.S. Pat. Nos. 3,657,744; 4,056,854; 5,411,552; 5,545,214; 5,855,601; 5,957,948; 6,458,153; 6,540,782; 7,510,575; 7,585,321; 7,682,390; and 9,326,856; and U.S. Pub. No. 2015/0320556.) Surgical or mechanical heart valves may be sutured into a native heart valve annulus of a patient during an open-heart surgical procedure, for example. Expandable heart valves may be delivered into a patient via a tube-like delivery apparatus such as a catheter, a trocar, a laparoscopic instrument, or the like to avoid a more invasive procedure such as full open-chest, open-heart surgery. As used herein, reference to an “expandable” heart valve includes those that are self-expending and those that are mechanically expandable via, for example, a balloon. Often the term “collapsible/expandable” heart valve is used herein and unless the text or the context dictate otherwise, this term is similarly meant to include heart valves that have a small cross-section that enables them to be delivered into a patient through a tube-like delivery apparatus in a minimally invasive procedure, and then self-expanded or mechanically expanded to an operable size once in place.

Prosthetic heart valves typically take the form of a one-way valve structure (often referred to herein as a valve assembly) mounted to/within a stent. In general, expandable heart valves include a self-expanding or balloon-expandable stent, often made of nitinol or steel. The one-way valve assembly mounted to/within the stent includes one or more leaflets, and may also include a cuff or skirt. The cuff may be disposed on the stent's interior or luminal surface, its exterior or abluminal surface, and/or on both surfaces. (See U.S. Pat. Nos. 6,458,153; 7,585,321; 8,992,608; 9,241,794; and 9,289,296; and U.S. Pub. No. 2015/0320556.) A cuff ensures that blood does not just flow around the valve leaflets if the valve or valve assembly are not optimally seated in a valve annulus. A cuff, or a portion of a cuff disposed on the exterior of the stent, can help retard leakage around the outside of the valve (the latter known as paravalvular or “PV” leakage).

Leaflets, cuffs and valve assemblies for prosthetic heart valves may be derived from various natural tissues or synthetic materials. Commercial natural tissues that have been chemically treated or “fixed” are often used. For example, leaflets could be made of bovine pericardium and cuffs could be made of porcine pericardium. (See, e.g., U.S. Pat. No. 5,957,949 at 6:23-33; U.S. Pat. No. 6,458,153 at 8:28-40; U.S. Pat. No. 5,855,601 at 6:21-30; and U.S. Pat. No. 7,585,321 at 13:5-36.) Other materials that may be used to form valve components include various synthetic polymers including, without limitation, polytetrafluoroethylene (PTFE) or polyester (see U.S. Pat. No. 5,855,601 at 6:29-31; U.S. Pat. Nos. 10,039,640; 10,022,211; 9,056,006; and 10,299,915; and U.S. Pub. Nos. 2018/0055632; 2017/0258585; 2018/0078368; and 2019/0201190), and elastic materials including silicone rubber and polyurethanes. (See U.S. Pat. No. 6,540,782 at 6:2-5.) These materials have been used in the form of continuous sheets, porous felts (U.S. Pat. No. 6,540,782 at 6:17-23) or woven fabrics. (See also U.S. Pat. Nos. 10,039,640; 10,299,915; 10,022,211; and 4,610,688; and U.S. Pub. Nos. 2018/0055632; 2017/0258585; and 2018/0078368; see also Basir et al., “Flexible mechanoprosthesis made from woven ultra-high-molecular-weight polyethylene fibers; proof of concept in a chronic sheep model”; Interactive CardioVascular and Thoracic Surgery, 25(2017) 942-949; Yamagishi and Kurosawa. “Outflow Reconstruction of Tetralogy of Fallot Using a Gore-Tex Valve;” Ann. Thorac Surg. 1993; 56:1414-17.) Valve components and valve assemblies may be attached to a collapsible/expandable stent or frame by sutures or may be molded, glued, or soldered to the stent. (See U.S. Pat. No. 7,585,321 at 13:30-31.)

Despite the disclosure of various natural tissues and synthetic materials for possible uses in various medical devices, little is often disclosed about the specifics of the structure and compositions of such elements beyond illustrations of their general structure and a generic identification of polymers that can be used. Those generalized disclosures show that, while the concept of polymer-based implantable medical devices and, in particular, valves, is known, actually successfully taking the broad concept to working solutions is far more challenging. Therefore, there exists a need for further improvements in the materials for these devices and the devices made therefrom.

### BRIEF SUMMARY OF THE DISCLOSURE

The disclosure describes single layer or multi-layered polymer sheet materials that may be used for construction of medical devices including, without limitation: venous valves, occluders, prosthetic vascular conduits, grafts, and embolic protection devices, sheets for treating hernias, skin patches, vaginal patches, cardiac patches, adhesion barriers, surgical heart valves (those requiring open chest surgery to implant) and expandable prosthetic heart valves which can be implanted using a catheter such as trans-femorally, trans-apically, and trans-septally. Structures made from the sheet materials in accordance with the present disclosure need not comprise the entire medical device but instead, parts or components of the device. For example, the sheet materials of the present disclosure could be used to produce one or more elements of a valve assembly used in expandable heart valves and surgical valves (aortic valves, mitral valves, pulmonary valves and tricuspid valves) such as interior cuffs, exterior cuffs, and/or leaflets.

The disclosure also describes and contemplates the medical devices made using these polymer sheet materials, as well as methods of making the sheet materials and the medical devices.

Polymer sheet materials in accordance with the present disclosure include a continuous or complete layer of a film or ply of polymer material which may be covered on a major surface and/or an edge with one or more additional layers and/or partial layers. As used herein, the terms “complete and continuous” or “continuous and complete” refer to a layer of material that extends throughout the dimensions of the sheet material being formed. Such layers may also be at times referred to as “continuous,” “complete” or “full” layers. Where a “continuous or complete” layer is the substrate to which all other layers or partial layers are applied, that layer is often referred to as the “primary” layer. The additional layers may be films or plies that are solid prior to their addition to the polymer sheet material or may be a coating applied in a fluid (solid, liquid or gas) form and dried, cooled or cured to form a solid. The sheet materials may, in some embodiments, be reinforced with individual or discrete fibers. In accordance with the present disclosure, however, polymer-containing sheet materials do not include woven or non-woven fabrics (knitted, felts, non-woven mats and the like) or fabric layers. The reinforcing fibers in a multi-layered sheet material may be oriented in different directions in different layers.

In some embodiments, the final or finished sheet material used in a medical device as described herein, whether the sheet material is composed of a single layer or multiple layers, will have a thickness of between about 1 μm and about 1,000 μm and in some embodiments, between about 1 μm and about 500 μm and in still others, between about 5 μm and about 300 μm. It also may have a tensile strength of at least about 35 N and in some embodiments at least about 50 N. In still other embodiments, the sheet material will have a tensile strength of at least about 70 N. In particular for the valve components of expandable or surgical valves, including leaflets and cuffs, the sheet materials used may exhibit one or more of the properties described in Table 1 below. It should be understood that, although Table 1 lists various characteristics with values grouped in a “broader range” and a “narrower range,” the sheet material may include any combination of characteristics from the “broader range” and the “narrower range,” and further, the sheet material may include in some instances characteristics that are outside the “broader range” and the “narrower range.”

TABLE 1

| Test Type/Characteristic | Test Method | Broader Range | | Narrower Range | |
|---|---|---|---|---|---|
| Thickness (Leaflets) | ASTM D1777-96 | 5 μm-500 μm | | 50 μm-350 μm | |
| Thickness (Cuffs) | ASTM D1777-96 | 1 μm-300 μm | | 5 μm-200 μm | |
| Thickness (Occluder) | ASTM D1777-96 | 1 μm-1,000 μm | | 1 μm-350 μm | |
| Ultimate tensile strength | ASTM D5035-11 | 1-500 | MPa | 25-250 | MPa |
| | ASTM D882-12 | | | | |
| Tear strength | ASTM D2261-13 | 5-100 | lbF | 10-40 | lbF |
| Permeability | ISO7198 | 1-2,000 | mL/cm²/min | 10-1,200 | |
| Suture Retention (where | ISO7198 | 10-100N | | 30-70N | |
| leaflet or cuff are attached | | | | | |
| via sutures | | | | | |
| Stiffness/Flexural Rigidity | ASTM D1388-14 | .001-8 | cm | .001-4 | cm |

| | | | |
|---|---|---|---|
| Stretch | ASTM D6614-07 | 1-400% | 3-50% |

In some embodiments, polymer sheet materials may comprise at least one polymer layer providing advantageous, improved or altered performance properties. These improved or altered performance properties may include, without limitation, one or more of: (1) adjusting surface roughness, (2) altering strength, abrasion resistance, and/or flexibility, (3) altering lubricity, (4) providing weight or rigidity to portions of the sheet material, (5) promoting folding in specific regions, (6) altering cell adhesion, (7) controlling cellular ingrowth; (8) resistance to thrombosis; and (9) retention or release of a therapeutic agent(s).

In one embodiment, a polymer sheet material includes at least one complete or continuous layer composed of UHMWPE. In another embodiment, this polymer sheet material is multilayered including at least one layer of UHMWPE, and in another, at least two layers of the multi-layered sheet material are composed of UHMWPE. In a further embodiment, the present disclosure contemplates a polymer leaflet or cuff for a prosthetic heart valve comprising a polymer sheet material including at least one complete or continuous layer composed of UHMWPE. In another embodiment, the polymer sheet material used for the leaflet or cuff is multilayered and at least one layer is UHMWPE, and in another, at least two of those layers are composed of UHMWPE.

In still a further embodiment, a leaflet or cuff produced using the UHMWPE-containing sheet materials just noted above includes: holes or grommets at tabs or adjacent attachment edges; rib(s); indicia; a partial layer on a major surface adjacent an edge and, in particular, an attachment edge and/or a free edge. Any of the foregoing can be reinforced with a polymer fiber or metal wire such as nitinol.

In some embodiments, UHMWPE-containing leaflets or cuffs may have one or more of: a thickness of about 250 μm or less, a tensile strength of at least about 75N and preferably at least about 90N; a stiffness/flexural rigidity of about 3.0+/−1.75 cm; a permeability of about 850-950 mL/cm2/min; a suture retention meeting ISO7198; a stretch/strain of about 20-25%; and a tear strength per meeting or exceeding ASTM D2261-13. Instead, or in addition, the leaflets or cuffs just described can be produced from a polyolefin such as PTFE. The overall properties can in some embodiments be similar to those noted earlier in this paragraph. The same is true of leaflets or cuffs made with a sheet material including a urethane.

The sheet material used for the leaflet or cuff (or any medical device or component thereof) can include a layer that is directionally oriented or that has directionally oriented properties. Where the sheet material used for the leaflet or cuff is multilayered, at least one layer may be directionally oriented. It is also possible that two or more of the layers could be directionally oriented, with the layers aligned so that their orientation directions are parallel with one another or biased at an angle relative to one another.

In one embodiment, a polymer sheet material includes at least one complete or continuous layer composed of a urethane or polyurethane. In another embodiment, this polymer sheet is multilayered, and in another, at least two layers are composed of urethane or polyurethane. In a further embodiment, the present disclosure contemplates a polymer leaflet or cuff for a prosthetic heart valve comprising a polymer sheet material including at least one complete or continuous layer composed of a urethane or polyurethane. In another embodiment, the polymer sheet used for the leaflet or cuff is multilayered, and in another, at least two layers are composed of urethane or polyurethane.

In still a further embodiment, a leaflet or cuff produced using the urethane or polyurethane sheet materials just noted above includes: holes or grommets at tabs or adjacent attachment edges; rib(s); indicia; a partial layer on a major surface adjacent an edge and, in particular, an attachment edge and/or a free edge. And any of the foregoing leaflets, cuffs or other structures of medical devices can be reinforced with a polymer fiber or metal wire such as nitinol.

Additional features and structures that can be incorporated in a single or multi-layered sheet material of the invention include, without limitation, stitches, suture lines, or wires. For example, a row of sutures could be added, of varying number of stitches, in a line or other desired shape, across the full length of the structure or any portion thereof. The properties can be altered based on the number and density of stitches, the number of sutures applied and the pattern in which they are applied. In one embodiment, by using one or more sutures extending from an attachment edge to a free edge of a leaflet, one can create areas of stiffening and more flexible zones or “hinges.” A suture could also be stitched to at least a portion of the attachment edge and/or the free edge of a leaflet to provide reinforcement and/or weight and/or to introduce or preserve a shape. And in still another aspect of this embodiment, wires, such as a steel or nitinol wire could be used and inserted on the surface of a layer, or between layers along the attachment edge, the free edge or across a major surface of a leaflet, again for an example. Wires or sutures or other structures could also be applied by stitching, gluing, laminating, etc. For example, a wire could be disposed between a sheet material and a coating or layer laminated thereto at the free edge of a leaflet. The wire or other reinforcement may extend across the entire edge, just a portion of it, may be continuous or discontinuous. One such wire or more than one can run in a semicircle or concentric semicircles spaced apart from the attachment edge and if more, from each other. Wires could also extend across the major surface of the leaflet from side to side. The wire could be bent to impart or preserve the three dimensional shape of a leaflet to preserve its “belly” or “spinnaker” shape in the leaflet.

In still a further embodiment, the medical device or an element thereof, could be constructed or attached so as to form a pleat or fold across its major surface or a portion thereof.

The sheet material used for the leaflet or cuff (or any medical device or component thereof), which includes at least one layer composed of urethane or polyurethane, can include a layer that is directionally oriented as previously noted.

Another embodiment of the disclosure provides methods of manufacturing a medical device, such as an expandable valve prosthesis, and/or producing components and assemblies therefor. These include various methods of making polymer sheet materials. These methods include mechanical methods, for example cutting with scissors or a blade. Other techniques include, for example, cautery, mechanical cutting, stamping, chemical, laser, ultrasonic, or water jet cutting, bio-glue, gluing, reacting, crosslinking, folding, sewing, riveting, spot welding, coating, 3D printing or laminating.

One embodiment includes a method of forming a leaflet for use in a prosthetic heart valve. The method includes cutting a leaflet-shaped structure from a polymer sheet material as described herein. Alternatively, the method includes forming a sheet material in the desired shape by molding a liquid or molten polymer material and allowing it to solidify into a sheet material in the desired shape. The method also includes layering a first layer of polymer material with one or more additional layer(s) and repeating the layering step until the leaflet has a selected thickness and desired physical and/or performance properties. Where the subsequent layers are preformed solid plies or films, these layers are affixed to one another by, for example, gluing, reacting, crosslinking, folding, sewing, riveting, spot welding or laminating. When the layer is added as a non-preformed coating, any known coating method may be used.

In an additional embodiment, the method of forming a leaflet for use in a prosthetic heart valve includes folding a first portion of a sheet material over a second portion of the sheet material and repeating the folding step until the leaflet has a selected thickness.

Another embodiment of the present disclosure provides a leaflet for use in a prosthetic heart valve including a pleat in a sewing region of the leaflet. This is shown in FIG. 30. During normal use of the valve, the pleat unfolds when a load is exerted on the leaflet as the leaflets coapt and reforms as the leaflets open. The pleat may be formed by folding a first portion of a polymer sheet over a second portion of the polymer sheet.

The sheet material and any medical device made using that sheet material may undergo sterilization. This may be done with one or more of a variety of sterilization modalities, for example, with ethylene oxide, peracetic acid, nitrogen oxide, e-beam, steam, gamma radiation, carbon dioxide and chemical liquid sterilant.

### BRIEF DESCRIPTION OF THE DRAWINGS

This disclosure may be best understood by reference to the following description, taken in conjunction with the accompanying drawing figures, in which:

FIG. 1A is a perspective view of a frame of a surgical prosthetic heart valve;

FIG. 1B is a perspective view of a sewing cuff insert of a surgical prosthetic heart valve;

FIG. 1C is a perspective view of the frame and sewing cuff insert of FIGS. 1A-B in an assembled condition;

FIG. 2 is a side view of a stent-supported prosthetic heart valve according to the prior art in an expanded condition;

FIG. 3 is a highly schematic transverse cross-section of the prosthetic heart valve taken along line 3-3 of FIG. 2 and implanted in a native valve annulus;

FIG. 4 is a highly schematic developed view of an expanded stent which is illustrated flattened as if it were cut longitudinally, illustrating inner and outer cuffs attached to the stent;

FIG. 5 is an exploded view of a complete or continuous primary polymer layer sandwiched between two further polymer film layers adhered to the opposed upper and lower major surfaces of the primary polymer layer;

FIG. 6 is a perspective view of a complete or continuous primary polymer layer having a partial layer on or adjacent the edges of the upper major surface of the primary polymer layer;

FIG. 7 is a perspective view of a complete or continuous primary polymer layer having a structured upper surface and a different number of additional polymer layers on each of the opposed major surfaces of the primary polymer layer;

FIG. 8A is a plan view of a leaflet covered with a partial layer on or adjacent the edges of a complete or continuous primary polymer layer;

FIG. 8B is a plan view of the underside of the leaflet covered with a partial layer along the sewing edge of a complete or continuous primary polymer layer;

FIG. 8C is a plan view of the top side of the leaflet covered with a partial layer along the free edge of a complete or continuous primary polymer layer;

FIG. 9 is a plan view of a heart valve leaflet fabricated from UHMWPE layers;

FIG. 10A is a longitudinal cross-section of a medical closure device according to an embodiment of the disclosure;

FIG. 10B is a highly schematic view of the medical closure device of FIG. 27A implanted into a left atrial appendage;

FIG. 11 is a schematic perspective view of a leaflet formed from a complete or continuous primary polymer layer according to the present disclosure;

FIG. 12 is a schematic perspective view of a leaflet formed from a complete or continuous primary polymer layer according to the present disclosure with a further layer affixed to a major surface thereof;

FIG. 13 is a schematic perspective view of a leaflet formed from a complete or continuous primary polymer layer with an additional layer affixed to each of the opposed upper and lower major surfaces of the primary polymer layer;

FIG. 14 is a schematic perspective view of a leaflet formed from a multilayered complete or continuous primary polymer layer with a different number of additional layers affixed to each of the opposed upper and lower major surfaces of the primary polymer layer;

FIG. 15 is a schematic perspective view of a leaflet formed from a multilayered complete or continuous polymer sheet material according to the present disclosure composed of two polymer layers directly affixed to one another over the majority of their respective opposed major surfaces, with a partial layer disposed between them at or adjacent the attachment edge of the leaflet;

FIG. 16 is a schematic perspective view of a leaflet formed from a multilayered complete or continuous polymer sheet material according to the present disclosure;

FIG. 17 is a schematic perspective view of a leaflet formed from a complete or continuous polymer sheet material according to the present disclosure including a partial layer affixed to or adjacent a free edge of a complete or continuous polymer layer;

FIG. 17A is a schematic partial cross-section of a stent and a valve assembly incorporating the leaflet of FIG. 17;

FIG. 18 is a schematic perspective view of a leaflet formed from a complete or continuous polymer sheet material according to the present disclosure including a partial layer affixed to or adjacent an attachment edge of a complete or continuous polymer layer;

FIG. 18A is a schematic partial cross-section of a stent and a valve assembly incorporating the leaflet of FIG. 18;

FIG. 19 is a schematic perspective view of a leaflet formed from a complete or continuous polymer sheet material according to the present disclosure including a first partial layer affixed to or adjacent a free edge of a first major surface of a complete or continuous polymer layer and another partial layer affixed to or adjacent an attachment edge of a second major surface of the complete or continuous layer;

FIG. 20 is a schematic perspective view of a leaflet formed from multiple complete or continuous polymer layers with a partial layer affixed to or adjacent a free edge thereof;

FIG. 21 is a schematic perspective view of a leaflet formed from a layered polymer sheet material having a partial layer forming ribs according to the present disclosure;

FIG. 22 is a schematic perspective view of a leaflet formed from a layered polymer sheet material having a partial layer forming ribs according to the present disclosure;

FIG. 23 is a schematic perspective view of a leaflet formed from a layered polymer sheet material having a partial layer forming spots according to the present disclosure;

FIG. 24 is a schematic perspective view of a leaflet formed from a complete or continuous polymer layer partially layered with different numbers of multiple partial coatings or partial films so as to form a leaflet of variable thickness;

FIG. 24A is a cross-sectional view of a variant of the leaflet of FIG. 41;

FIG. 24B is a cross-sectional view of a further variant of the leaflet of FIG. 41;

FIG. 25 is a schematic perspective view of a leaflet formed from a complete or continuous polymer layer partially layered on its upstream major surface according to the present disclosure;

FIG. 26 is a schematic perspective view of a leaflet formed from a multilayered polymer sheet material incorporating indicia according to the present disclosure;

FIG. 26A is a highly schematic transverse cross-section of a prosthetic heart valve incorporating a plurality of the leaflets of FIG. 26;

FIG. 27 is a schematic perspective view of a leaflet formed from a polymer sheet material incorporating indicia according to the present disclosure;

FIGS. 27A-27C are highly schematic transverse cross-sections of a prosthetic heart valve incorporating a plurality of the leaflets of FIG. 27 with different indicia;

FIG. 28 is a schematic perspective view of a leaflet formed from a complete or continuous polymer layer including a partial layer affixed to or adjacent an attachment edge of the complete or continuous layer, the leaflet incorporating holes or grommets according to the present disclosure;

FIG. 28A is a schematic partial cross-section of a stent and a valve assembly including a cuff and the leaflet of FIG. 28;

FIG. 28B is a schematic partial cross-section of a stent and a valve assembly incorporating the leaflet of FIG. 28;

FIG. 29 is a schematic perspective view of a stent having a cuff formed from a multilayered polymer sheet material incorporating radiographic bands according to the present disclosure;

FIG. 29A is a schematic partial cross-section of the stent and cuff of FIG. 29;

FIG. 30 is a schematic view of a leaflet formed from a multilayered polymer sheet material incorporating pleats according to the present disclosure;

FIG. 31 is a schematic perspective view of a leaflet formed from a sheet material according to the present disclosure including a single stitch in a major surface of the leaflet;

FIG. 32 is a schematic perspective view of a leaflet formed from a sheet material according to the present disclosure including a single stitch in the free edge of the leaflet;

FIG. 33 is a schematic perspective view of a leaflet formed from a sheet material according to the present disclosure including multiple stitches on a major surface of the leaflet;

FIG. 34 is a schematic perspective view of a leaflet formed from a sheet material according to the present disclosure including multiple stitches in the free edge of the leaflet;

FIG. 35 is a schematic perspective view of a leaflet formed from a sheet material according to the present disclosure including a suture line extending across a major surface of the leaflet from the attachment edge to the free edge;

FIG. 36 is a schematic perspective view of a leaflet formed from a sheet material according to the present disclosure including a plurality of suture lines extending across a major surface of the leaflet from the attachment edge to the free edge;

FIG. 37 is a schematic perspective view of a leaflet formed from a sheet material according to the present disclosure including a discontinuous suture line extending across a major surface of the leaflet from the attachment edge to the free edge;

FIG. 38 is a schematic perspective view of a leaflet formed from a sheet material according to the present disclosure including a suture line extending along the free edge of the leaflet;

FIG. 39 is a schematic perspective view of a leaflet formed from a sheet material according to the present disclosure including a discontinuous suture line extending along the free edge of the leaflet;

FIG. 40 is a schematic perspective view of a leaflet formed from a sheet material according to the present disclosure including a suture line extending along the attachment edge of the leaflet;

FIG. 41 is a schematic perspective view of a leaflet formed from a sheet material according to the present disclosure including a discontinuous suture line extending along the attachment edge of the leaflet;

FIG. 42 is a schematic perspective view of a leaflet formed from a sheet material according to the present disclosure including a suture line extending along the free edge of the leaflet through a partial coating layer;

FIG. 43 is a schematic perspective view of a leaflet formed from a sheet material according to the present disclosure including a suture line extending along the free edge of the leaflet disposed underneath a partial coating layer;

FIG. 44 is a schematic perspective view of a leaflet formed from a sheet material according to the present disclosure including a wire extending from the attachment edge to the free edge of the leaflet;

FIG. 45 is a schematic perspective view of a leaflet formed from a sheet material according to the present disclosure including a discontinuous wire extending from the attachment edge to the free edge of the leaflet;

FIG. 46 is a schematic perspective view of a leaflet formed from a sheet material according to the present disclosure including a wire extending across a major surface of the leaflet and along the free edge of the leaflet, the wire being disposed between a complete or continuous layer and a partial coating layer;

FIG. 47 is a schematic perspective view of a leaflet formed from a sheet material according to the present disclosure including a wire extending across a major surface of the leaflet and generally along the attachment edge of the leaflet;

FIG. 48A is a schematic perspective view of a leaflet formed from a sheet material according to the present disclosure including a wire extending across a major surface of the leaflet and generally along a line which parallels the leaflet's free edge; and

FIG. 48B is a schematic perspective view of a leaflet formed from a sheet material according to the present disclosure including a plurality of concentric semicircular wires roughly paralleling the attachment edge.

### DETAILED DESCRIPTION

As used herein in connection with a prosthetic heart valve, the term “inflow end” refers to the end of the heart valve through which blood enters when the valve is functioning as intended, and the term “outflow end” refers to the end of the heart valve through which blood exits when the valve is functioning as intended. As used herein, the terms “proximal” and “upstream” refer to the inflow end of a prosthetic heart valve and these terms may be used interchangeably. The terms “distal” and “downstream” refer to the outflow end of a prosthetic heart valve and also may be used interchangeably. As used herein, the terms “generally,” “substantially,” and “about” are intended to mean that slight deviations from absolute are included within the scope of the term so modified. When used herein in the context of a prosthetic heart valve, or a component thereof, the lengthwise or axial direction refers to the direction parallel to a longitudinal axis passing through the center of the stent or heart valve from the inflow end to the outflow end. When used herein in the context of a prosthetic heart valve, or a component thereof, the circumferential direction refers to the direction extending along the circumference of the prosthetic heart valve.

“Nonporous polymer sheet material(s),” “nonporous polymer-containing sheet material(s),” “polymer sheet material(s),” “sheet material(s),” “sheet(s),” “polymer sheets,” and similar expressions used in this document are used interchangeably unless the context suggests otherwise, or the disclosure indicates otherwise. Unless specified otherwise, these terms are meant to embrace a preformed solid sheet of one or more polymer materials comprising a single layer or multiple layers from which a medical device or component thereof can be made. Polymer sheets have at least one continuous or complete polymer layer with two opposed major surfaces and at least one side edge. A polymer sheet that has the shape of a piece of printer paper has two major surfaces and four side edges. Cutting a circle from that sheet would result in a structure still having two major surfaces but a single side edge. A multiple layer sheet is formed by adding one or more complete or partial layers to at least one major surface of the continuous or complete layer. These may be affixed to each other by any known method including, without limitation, being formed, glued, laminated together or otherwise attached to one another to form a single, albeit composite, structure. By analogy, a multilayered polymer sheet can be thought of as a ream of sheets of copy or printer paper, composed of multiple superimposed layers (each sheet of paper), the collection having two major surfaces—the outwardly facing surfaces of the top and bottom layers, respectively, and four side edges formed by the composite or collection of the side edges of the individual sheets of paper within the ream. To complete the analogy, the individual sheets of paper within the ream would have been glued or otherwise attached together. These complete or continuous polymer sheet materials may be layered with one or more partial layers affixed to a major surface or side edge of the sheet material or a structure made therefrom.

Polymer sheets disclosed and used to form medical devices and components thereof include at least one layer. “Layer” means, as the context permits, a solid preformed ply or film, or a coating on a pre-formed ply or film used to form a polymer sheet. Layers need not be coextensive in terms of area or thickness. There may be “partial layers”—layers that do not cover an entire major surface of a continuous or complete layer to which the partial layer(s) are attached. For example only, in a stacked structure of multiple layers, the topmost layer could cover only 50% of a major surface of the layer disposed immediately below or above it. A topmost layer could be applied to a small portion of the top major surface of the next layer below it, wrap around a portion of the side edge, and also be applied to a small portion of the otherwise outermost major surface of the bottom layer, thereby covering the portion of the side edge. Layers could be applied in a discontinuous manner to form a pattern or relief on the layer to which they are applied, and multiple smaller layers could be applied having different shapes, sizes and thicknesses. A partial layer could be sandwiched between two complete or continuous layers. As one example, a partial layer could be disposed between two layers adjacent the attachment edge of a leaflet to reinforce it at its point of attachment to a cuff and/or stent. As a second example, a partial layer which tracks all of the edges of a leaflet could be sandwiched between two complete or continuous layers such that the center of the leaflet is composed of only two layers but the periphery of the leaflet is composed of three layers. This can provide extra strength to the leaflet while limiting weight and providing additional flexibility in its center.

As noted earlier, a layer can be composed of a “coating” that is applied as a fluid (a liquid semi solid or solid particles) to a major surface or edge of a preformed solid film or ply. The coating can cool, dry, cure, de-solvate or otherwise be formed into a solid layer on a surface of the film or ply (including being formed on a complete or continuous layer or a partial layer, whether formed from a film or a prior coating). A coating can be vapor deposited, deposited as a plasma, or plated onto a surface of a sheet. It can be sprayed on, painted on and/or result from being dipped. Coatings may be made of a polymer as defined herein but need not be. While a coating is often composed of one or more of the polymers noted elsewhere herein, a coating could be composed of a metal, such as silver, gold, steel, nitinol or copper A coating may also be composed of biological materials such as, without limitation, blood proteins, blood plasma, coagulation products, fibrin or other materials or drugs or pharmacologically active agents.

In some embodiments, layers, whether films or coatings, may provide, improve or alter performance properties, including one or more of: (1) adjusting surface roughness, (2) altering strength, abrasion resistance, and/or flexibility, (3) altering lubricity, (4) providing weight or rigidity to portions of the sheet material, (5) promoting folding in specific regions, (6) altering cell adhesion, (7) controlling cellular ingrowth; (8) resistance to thrombosis; and (9) retention or release of a therapeutic agent(s). The sheet material may also be contoured, scored, or patterned. By using various layers and partial layers, one or more surfaces of the polymer sheet material or structure made therefrom can be contoured or patterned by having a major surface covered by one or more layers of various thicknesses and compositions. This can be accomplished in several ways. A variety of partial layers of different thicknesses, a different number of layers, etc., may be applied to one or major surfaces of a complete or continuous primary layer. This also can be accomplished by applying multiple coating layers to provide specific structural features to a side, edge or surface of the primary layer. And a mixture of coatings and films can be used to contour or pattern a surface of the primary layer. Patterns and contours may also be created in layers and/or surfaces by removing or ablating a portion thereof.

“Polymer” and similar terms as used in connection with the sheet materials of the disclosure include any polymer, or any mixture of polymers and/or copolymers that is sufficiently flexible so as to be useful to the particular structure being made therefrom, sufficiently durable, and biologically acceptable for implantation. For example, a leaflet used in an expandable heart valve such as a transcatheter aortic valve replacement (TAVR) or transcatheter mitral valve replacement (TMVR) must be sufficiently flexible and sufficiently durable to permit it to open and close hundreds of millions of times.

Polymers may include, without limitation, polyolefins such as halogenated polyolefins such as polytetrafluoroethylene (PTFE) (which includes expanded and stretched PTFE and PTFE of any molecular weight) (also known as Teflon®), polyethylenes including those of any molecular weight (e.g., ultra-high molecular weight polyethylene (UHMWPE), e.g., having an average molecular weight of between about 2 and about 7 5 million atomic mass units), and polypropylenes including those of any molecular weight (e.g., ultra-high molecular weight polypropylene (UHMWPP)). Other polymers which may be used include urethanes including polyurethanes, polyether ether ketone (PEEK), polyvinyl alcohols, silicones, rayons, polyesters, aramids, acrylates, and spandex. Homo- and co-polymers of these materials may be used. Additionally, blends of polymers and/or copolymers may be used to form layers. See U.S. Patent Pub. No. 2018/0055631 A1, the text of which regarding the structure, function and manufacture of a heart valve are hereby incorporated by reference herein. In some embodiments, the polymer used for the sheet materials, including continuous or complete polymer layer(s), partial layer(s) and/or complete or partial coating(s), can be bioabsorbable, biodegradable, and/or bio-erodible. Exemplary bioabsorbable, biodegradable, and/or bio-erodible polymers may include poly-glycolic acid, poly-L-lactic acid, copolymers of poly-glycolic acid, poly-L-lactic acid, polycaprolactone, poly-DL lactic acid, polytrimethylene carbonate, polydioxanone, poliglecaprone and polyglactinas well as blends, mixtures and copolymers of the foregoing. Using a bio-absorbable/biodegradable polymer for a layer could, for example, retard cell attachment until the layer erodes or is absorbed.

In another embodiment, and as described elsewhere herein, the layer may include an active pharmaceutical ingredient (API) that is released gradually. Taxol and other drugs have been released from coated stents in a like manner for a variety of reasons, including mitigating the initial stress of placement of the stent. But it may be that a multilayered sheet material as disclosed herein is in contact with the annulus of a native heart valve, for example. Using a thin outermost layer of a cuff material of the disclosure, for example made of one or more bio-absorbable/biodegradable polymers, could facilitate drug release, and then dissolve. APIs may include, for example, Sirolimus, Paclitaxel (Taxol), Zotorolimus or Everolimus, or any treatment to enhance resistance to calcification. APIs may also include growth factors, such as vascular endothelial growth factor (VEGF) and transforming growth factor (TGF-beta). It may also include hyaluronan, hyaluronic acid, glycosaminoglycans (GAGs), Heparin, or amino acids for cell attachment sites, and antioxidants such as super oxide dismutase or ascorbic acid.

The polymer or polymers used may be selected based on certain properties, such as creep, tensile strength, elastic modulus, strain/elongation, compressibility, flexural rigidity and stiffness. Other properties that may influence the selection of polymers include melt flow viscosity, linear density, tenacity, melting temperature, biocompatibility, purity, color, radiopacity, surface friction and entanglement. Still other polymer properties that may be sought include a specific anisotropy, color, weight, extractable content, permeability, radiation sensitivity, radiopacity, moisture sensitivity, temperature sensitivity, and/or chemical sensitivity. Many of these parameters may be influenced by the particular polymer material used to form the sheet materials, while others may be more influenced by the manner in which the sheet materials are formed.

“Nonporous” means having a permeability as noted in the broad range recited in Table 1.

The polymer sheet materials of the present disclosure may be “reinforced.” Reinforcement herein means employing reinforcing structures, such as fibers or other structures, to increase the rigidity, strength, structural integrity and/or durability of a layer and a sheet material made therefrom. The reinforcing structures can be embedded within a single polymer layer or can be disposed between successive layers, or both. If fibers are used, they may all extend in substantially the same direction so as to not be intertwined, or they may be present in an amount of 50 fibers per square inch or less, and in some embodiments 20 fibers per square inch or less, and in still other embodiments, 10 per square inch or less. Often, the fibers are spaced apart and oriented in the same direction. The fibers can be made of metal, such as stainless steel, copper, shape-memory metals such as nitinol (a nickel-titanium alloy), and the like; polymers or other synthetic materials, such as UHMWPE or polyurethane; or natural materials, such as cotton or hemp. The fibers may have a variety of diameters which may depend on the material from which the fibers are formed, as well as their purpose. Typically, for fibers embedded within a polymer layer, the diameter of the fibers will be less than the thickness of the polymer layer in which the fibers are embedded. For example, where the fibers are formed from nitinol, the nitinol fibers, in some embodiments, may have a diameter of between about 0.0005 inches and about 0.0016 inches. In other embodiments, the nitinol fibers may have a diameter of between about 0.0009 inches and about 0.0015 inches. In some embodiments, one or more of the reinforcing fibers may be radiopaque.

“Reinforced” or “reinforcing” layers refers to layers as defined herein that are applied to at least a portion of a major surface and/or edge of a continuous or complete primary layer to provide rigidity, strength, durability and/or structural integrity thereto. The reinforcing structures could also be embedded in a layer—such as by employing a film that incorporates reinforcing fibers or placing reinforcing fibers in the desired position, pattern and/or orientation and then applying a coating material thereover to form a layer. Reinforcing structures can also be disposed between adjacent layers.

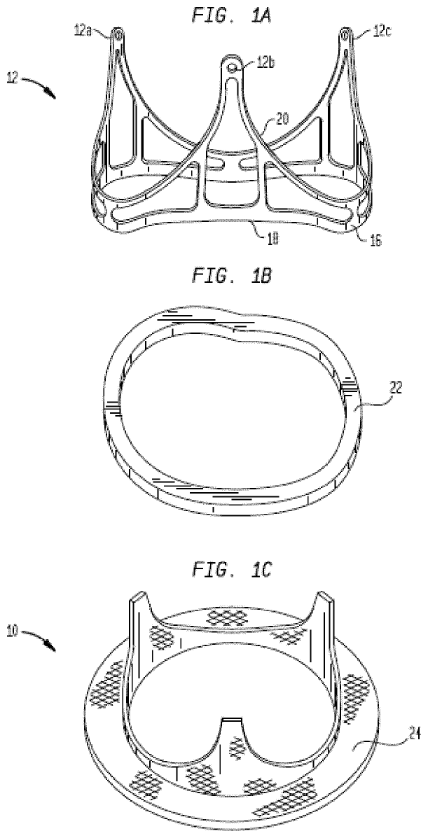

FIGS. 1A-1C illustrate a conventional surgical heart valve 10 and several components thereof. Surgical heart valve 10 may be surgically implanted into a patient to replace a native heart valve that may not be functioning as intended, such as the aortic valve, mitral valve, pulmonary valve, or the tricuspid valve. Surgical heart valve 10 may have a non-collapsible frame 12, shown in FIG. 1A, having a generally annular shape. Frame 12 may be formed of any suitable biologically compatible material, including titanium, Elgiloy® MP3N, or another metal, which may be laser cut from a tube, or from a biologically compatible polymer, such as PEEK or acetal. Since the valve of the illustrative embodiment is a tricuspid valve (e.g., for use in replacing a patient's aortic valve), frame 12 has three commissure posts 12a, 12b, and 12c that are equally spaced from one another around the circumference of the frame. Each commissure post stands up from the annularly continuous base 16 of frame 12, and they support and/or serve as attachment points for a plurality of prosthetic leaflets (not shown). Although frame 12 is illustrated with three commissure posts 12a-c for supporting a three-leaflet valve assembly, it should be understood that the frame could include more or fewer commissure posts for supporting a corresponding number of prosthetic leaflets. Base 16 of frame 12 may include a blood-inflow edge 18 that is scalloped as one proceeds around the frame to approximately match the natural scallop of the native valve annulus. The frame may also include an annularly continuous blood-outflow edge 20, which merges with and becomes part of each commissure post 12a-c . The inflow edge 18, outflow edge 20, and flexibility of the frame are designed to help ensure proper opening and coaptation of the leaflets of the prosthetic heart valve during use. The prosthetic leaflets may be formed of a biological material, such as bovine pericardium, or from any of the engineered leaflet materials disclosed herein.

Frame 12 may be covered by a fabric covering (not shown), particularly over each commissure post 12a-c . One example of an appropriate covering fabric is a spun form of polyester. A ring 22 (FIG. 1B), which may be formed of silicone, may be positioned around the outside of the inflow edge 18 of frame 12. The entire frame 12 and ring 22 may be completely covered inside and out by a further fabric or sheet layer. Subsequently, a layer of tissue 24 may be applied over the fabric or sheet layer, including both inside and outside of frame 12 and over ring 22. Tissue layer 24 is typically formed of any mammalian tissue, and in particular any mammalian pericardium tissue, such as porcine, equine, or bovine pericardium. In the completed surgical heart valve 10, the covered ring 22 serves as a sewing cuff for sewing the prosthetic heart valve into the native valve annulus of the patient.

The collapsible/expandable prosthetic heart valves of the disclosure have an expanded condition and may also have a collapsed condition. Although aspects of the disclosure apply to a collapsible/expandable prosthetic heart valve for replacing a native aortic valve, the disclosure is not so limited, and may apply to prosthetic valves for replacing other types of cardiac valves, including, the mitral valve, the tricuspid valve and the pulmonary valve. Nor is the disclosure limited to a specific method of delivery. For example, the collapsible/expandable prosthetic heart valves described herein may be delivered via any suitable transcatheter delivery route, including a transfemoral route, a transvenous route, a transapical route, a transjugular route, a transaortic route, a transsubclavian route, etc. Further, the collapsible/expandable prosthetic heart valves described herein may be delivered via traditional surgical routes, or any suitable minimally invasive route.

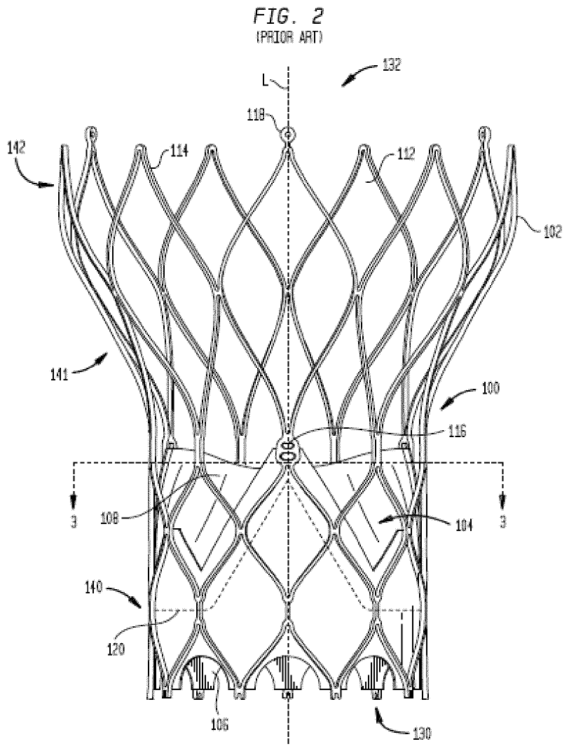

FIG. 2 shows one embodiment of a collapsible/expandable stent-supported prosthetic heart valve 100 according to the prior art, the prosthetic heart valve being shown in an expanded condition. Prosthetic heart valve 100 is designed to replace the function of the native aortic valve of a patient and includes a stent 102 which serves as a frame for the valve elements. Stent 102 extends along a lengthwise or longitudinal axis L from an inflow or annulus end 130 to an outflow or aortic end 132, and includes an annulus section 140 adjacent inflow end 130 and an aortic section 142 adjacent outflow end 132. Annulus section 140 may be in the form of a cylinder having a substantially constant diameter along its length, and may have a relatively small transverse cross-section in the expanded condition in comparison to the transverse cross-section of aortic section 142. A transition section 141 may taper outwardly from annulus section 140 to aortic section 142. Each of the sections of stent 102 includes a plurality of cells 112 formed by interconnected struts 114. Each cell 112 may include four struts 114 connected together generally in a diamond shape so as to form a cell that may be readily collapsed and expanded. It will be appreciated that a smaller or larger number of struts may be used to form cells having a different shape. The cells 112 in each section of stent 102 may be connected to one another in one or more annular rows around the stent. For example, as shown in FIG. 2, annulus section 140 may have two annular rows of complete cells 112, with the cells in one annular row offset by one-half cell width in the circumferential direction from the cells in the other annular row. Aortic section 142 and transition section 141 may each have one or more annular rows of complete or partial cells 112. The cells in aortic section 142 may be larger than the cells in annulus section 140 so as to better enable prosthetic valve 100 to be positioned within the aortic annulus without the structure of stent 102 interfering with blood flow to the coronary arteries. At least partly due to the shape of cells 112, stent 102 elongates in the direction of longitudinal axis L as the cells collapse when the stent transitions from the expanded condition to the collapsed condition, and shortens in the direction of longitudinal axis L as the stent transitions from the collapsed condition to the expanded condition.

Stent 102 may include one or more retaining elements 118 at outflow end 132, the retaining elements being sized and shaped to cooperate with retaining structures provided on a delivery device (not shown). The engagement of retaining elements 118 with the retaining structures on the deployment device may help maintain prosthetic heart valve 100 in assembled relationship with the deployment device, minimize longitudinal movement of the prosthetic heart valve relative to the deployment device during unsheathing or resheathing procedures, and help prevent rotation of the prosthetic heart valve relative to the deployment device as the deployment device is advanced to the target location and during deployment. One such deployment device is described in U.S. Patent Publication No. 2012/0078352, the disclosure of which is hereby incorporated by reference herein.

Stent 102 may also include a plurality of commissure attachment features 116 for mounting the leaflet commissures of the valve assembly to the stent. As can be seen in FIG. 2, each commissure attachment feature 116 may lie at the intersection of four cells 112, two of the cells being adjacent one another in the same annular row, and the other two cells being in different annular rows and lying in end-to-end relationship. Commissure attachment features 116 may be positioned entirely within annulus section 140 or at the juncture of annulus section 140 and transition section 141, and may include one or more eyelets or apertures which facilitate the suturing of the leaflet commissures to stent 102. Stent 102 may be formed as a unitary structure, for example, by laser cutting or etching a tube of a superelastic and/or shape-memory metal alloy, such as nitinol. It should be understood that stent 102 may include other forms of commissure attachment features, or may omit commissure attachment features, with the prosthetic leaflets being attached to the stent via other mechanisms, such as direct suturing or via intermediary attachment panels. Examples of other attachment modalities may be found in U.S. Patent Pub. No. 2020/0093590, the disclosure of which is hereby incorporated by reference herein. Stents may be continuous or discontinuous (made in sections that are attached to one another directly or indirectly). See, for example, U.S. Pat. No. 5,957,949.

Prosthetic heart valve 100 includes a valve assembly 104 which, in one embodiment, may be positioned entirely in the annulus section 140 of stent 102. Valve assembly 104 includes a plurality of leaflets 108 that collectively function as a one-way valve by coapting with one another, and a cuff 106 that may be positioned on the luminal surface of stent 102 surrounding leaflets 108. Although cuff 106 is shown in FIG. 2 as being disposed on the luminal or inner surface of annulus section 140, the cuff may be disposed on the abluminal or outer surface of the annulus section, or may cover all or part of either or both of the luminal and abluminal surfaces of the annulus section. As prosthetic heart valve 100 is intended to replace the aortic valve (which ordinarily is a tri-leaflet valve), it is shown in FIG. 2 with three leaflets 108. Adjacent leaflets 108 join one another at leaflet commissures. Each of the leaflet commissures may be sutured to a respective one of the three commissure attachment features 116. Between the leaflet commissures, each leaflet 108 may be sutured to stent 102 and/or to cuff 106 along an attachment edge 120, indicated with broken lines in FIG. 2. Leaflets 108 may be joined to stent 102 and/or to cuff 106 by techniques known in the art other than suturing. Above attachment edge 120, leaflets 108 are free to move radially inward to coapt with one another along their free edges. When prosthetic heart valve 100 is implanted in the native aortic valve annulus, blood flows in an antegrade direction from inflow end 130, past leaflets 108, and toward outflow end 132. This occurs when the pressure in the left ventricle is greater than the pressure in the aorta, forcing leaflets 108 to open. When the pressure in the aorta is greater than the pressure in the left ventricle, leaflets 108 are forced closed and coapt with one another along their free edges, blocking blood from flowing through prosthetic heart valve 100 in a retrograde direction from outflow end 132 to inflow end 130, which allows the left and right coronary arteries to fill and feed blood to the heart muscle. It will be appreciated that prosthetic heart valves according to aspects of the present disclosure may have more or less than the three leaflets 108 and commissure attachment features 116 shown in FIG. 2 and described above.

Cuff 106 may be scalloped at the inflow end 130 of stent 102, and may have a zig-zag structure at its outflow end, following certain stent struts 114 up to commissure attachment features 116 and other stent struts closer to the inflow end of the stent at circumferential positions between the commissure attachment features. When open, leaflets 108 may remain substantially completely within annulus section 140, or they may be designed to extend into transition section 141. In the embodiment shown, substantially the entirety of valve assembly 104 is positioned between the inflow end 130 of stent 102 and commissure attachment features 116, and none of the valve assembly is positioned between the commissure attachment features and the outflow end 132 of the stent.

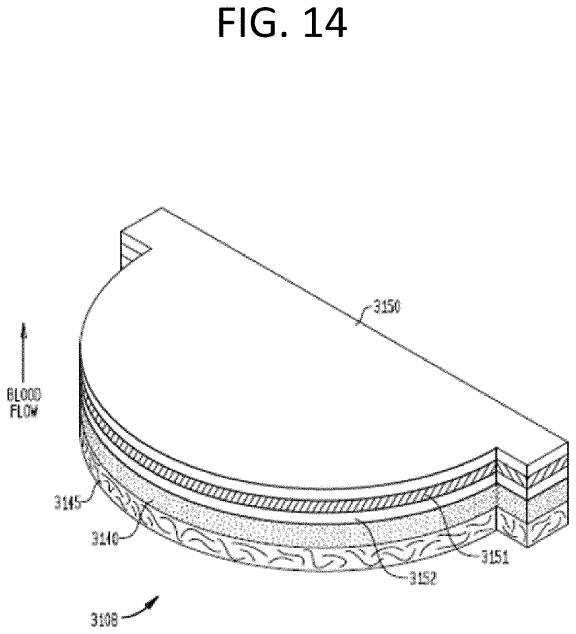

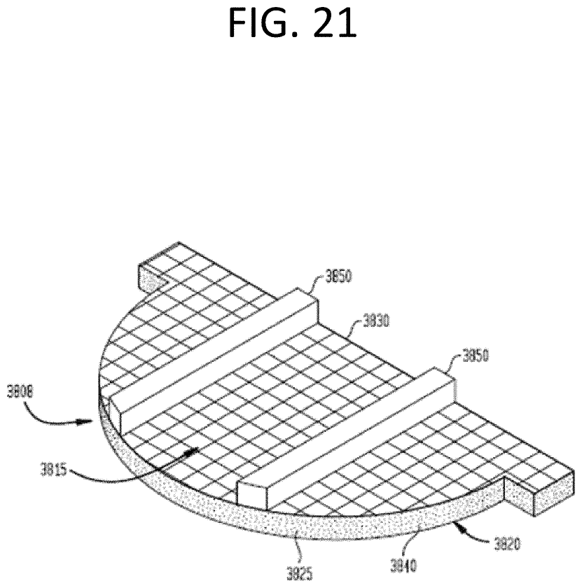

In operation, prosthetic heart valve 100 may be used to replace a native heart valve, such as the aortic valve, a surgical heart valve, or a heart valve that has undergone a surgical procedure. Prosthetic heart valve 100 may be delivered to the desired site (e.g., near the native aortic annulus) using any suitable delivery device. During delivery, prosthetic heart valve 100 is disposed inside the delivery device in the collapsed condition. The delivery device may be introduced into the patient using any known percutaneous procedure, such as a transfemoral, transapical, transvenous, or transseptal delivery procedure. Once the delivery device has reached the target site, the user may deploy prosthetic heart valve 100. Upon deployment, prosthetic heart valve 100 expands into secure engagement within the native aortic annulus. When prosthetic heart valve 100 is properly positioned inside the heart, it works as a one-way valve, allowing blood to flow in one direction and preventing blood from flowing in the opposite direction. (See U.S. Pat. No. 7,585,321 FIGS. 13a-16b and accompanying disclosure; U.S. Pat. No. 6,458,153 FIGS. 20A-20I and accompanying disclosure.)

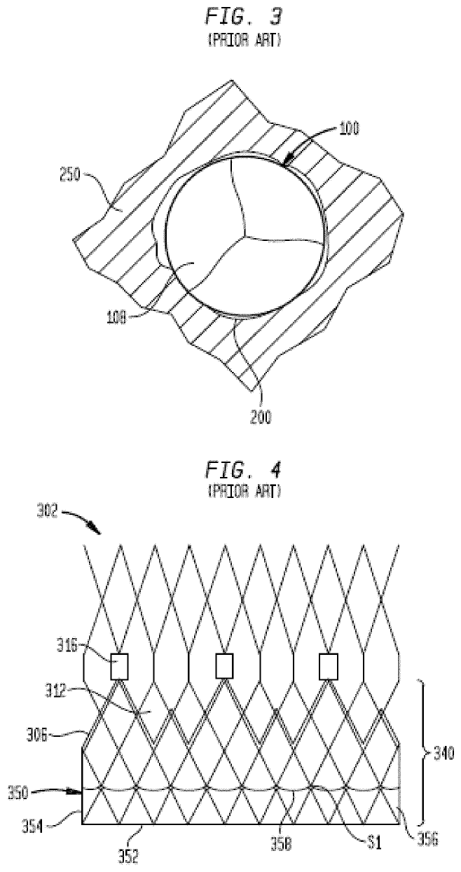

FIG. 3 is a highly schematic transverse cross-section of prosthetic heart valve 100 taken along line 3-3 of FIG. 2 and showing leaflets 108 disposed within native valve annulus 250. As can be seen, the substantially circular annulus section 140 of stent 102 is disposed within a non-circular native valve annulus 250. At certain locations around the perimeter of prosthetic heart valve 100, gaps 200 are formed between the heart valve and native valve annulus 250. Retrograde blood flow through these gaps and around the outside of the valve assembly 104 of prosthetic heart valve 100 can result in PV leak or regurgitation and other inefficiencies which can reduce cardiac performance Such improper fitment may be due to suboptimal native valve annulus geometry, for example, as a result of the calcification of the tissue of native valve annulus 250 or the presence of unresected native leaflets.

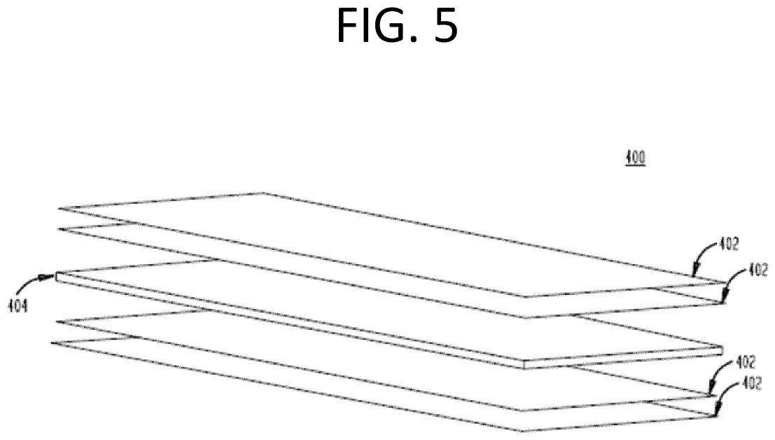

FIG. 4 depicts a collapsible/expandable prosthetic heart valve very similar to that shown in FIGS. 2 and 3, except that it is shown as if cut longitudinally and flattened. The heart valve can include a stent 302 with commissure attachment features 316. A cuff 306 similar or identical to cuff 106 may be positioned on the luminal and/or abluminal surface of stent 302. Indeed, cuff 306 in FIG. 4 is illustrated as being positioned on the luminal or inner surface of stent 302. However, in order to help minimize or eliminate PV leak, for example through the gaps 200 shown in FIG. 3, additional material may be coupled to the exterior of stent 302 as an outer cuff 350. In the illustrated example, outer cuff 350 may have a substantially rectangular shape and may be wrapped around the circumference of stent 302 at the inflow end of the stent so as to overlap in the longitudinal direction of the stent with cuff 306. This is only one embodiment of such an exterior or outer cuff. Outer cuff 350 may be formed as a single piece of material having a proximal edge 352, two side edges 354, 356, and a distal edge 358. Preferably, the proximal edge 352 of outer cuff 350 is coupled to stent 302 and/or to inner cuff 306 at or near the inflow end of the stent, for example by a continuous line of sutures (not shown), with the side edges 354 and 356 of the outer cuff joined to one another, so that retrograde blood flow (flowing from the outflow end toward the inflow end) entering the space between the outer cuff and the inner cuff cannot pass in the retrograde direction beyond the combined cuffs. In order to allow retrograde blood flow to enter the space between outer cuff 350 and inner cuff 306, the distal edge 358 of the outer cuff may be attached to stent 302 and/or to inner cuff 306 at locations that are spaced apart in the circumferential direction. The distal edge 358 of outer cuff 350 may, for example, be sutured to stent 302 at attachment points 51 located where each cell 312 in the proximal-most row of cells intersects with an adjacent cell in that same row. In the illustrated example, since there are nine cells 312 in the proximal-most row, there are nine separate attachment points 51 at which the distal edge 358 of outer cuff 350 is sutured or otherwise attached to stent 302 and/or to inner cuff 306. Retrograde blood flow around the abluminal surface of stent 302 may enter the pocket or space between outer cuff 350 and inner cuff 306 via the spaces between adjacent attachment points S1. Once retrograde blood flow enters this space, outer cuff 350 may tend to billow outwardly, helping to fill any of gaps 200 between the prosthetic heart valve and native valve annulus 250. Although the foregoing description uses the term “inner” in connection with cuff 306, that is merely intended to indicate that cuff 306 is positioned radially inward of outer cuff 350. Inner cuff 306 may be located either on the luminal or abluminal side of stent 302, or on both sides.

Outer cuff 350 may also comprise multiple pieces of material that, when joined together, form a shape and provide a function that are similar to what has been described above. Also, rather than being formed of a single substantially rectangular piece of material that is wrapped around the circumference of stent 302, outer cuff 350 may be formed as a continuous annular web without side edges 354, 356. Preferably, outer cuff 350 has an axial height measured from its proximal edge 352 to its distal edge 358 that is approximately half the axial height of a cell 312 in the proximal-most row of cells in stent 302 as measured along the major axis of the cell between two of its apices when the cell is in an expanded condition. However, outer cuff 350 may have other suitable heights, such as the full axial height of a cell 312 in the proximal-most row of cells, or more or less than the full axial height of a cell 312 in the proximal-most row of cells. Still further, although inner cuff 306 and outer cuff 350 are described above as separate pieces of material joined to stent 302 and to each other, the cuffs may be formed integrally with one another from a single piece of material that is wrapped around the inflow edge of the stent, with the distal edge 358 of the outer portion of the cuff joined to the stent and/or to the inner portion of the cuff at attachment points 51 as described above. With this configuration, the proximal edge 352 of outer cuff 350 does not need to be sutured to stent 302, although it still may be preferable to provide such attachment. The various valve components including, without limitation, inner cuffs, outer cuffs and leaflets, and valve assemblies made therefrom, may be attached to each other and/or to the stent in any conventional manner, including suturing, gluing, molding, welding, heating, cross-linking, and the like. (See U.S. Pat. Nos. 6,821,297; 6,458,153; 7,585,321; 5,957,949.)

Valve assemblies, such as valve assembly 104 comprising inner cuff 106/306, leaflets 108, as well as outer cuff 350, may be formed of the same or different materials, including any suitable biological material, including “fixed” bovine or porcine tissue, or a polymer such as, for example, polyolefins such as polytetrafluoroethylene (PTFE), polyethylenes including ultra-high molecular weight polyethylene (UHMWPE), and polypropylene, as well as polyurethane, PEEK, polyvinyl alcohol, silicone, or combinations thereof. (See U.S. Pub. No. 2018/0055631 A1, the disclosure of which regarding the structure, function and manufacture of a heart valve are hereby incorporated by reference herein.) In accordance with the present disclosure, at least one of the components of a valve, including, without limitation, leaflets or cuffs, valve assemblies, and the like, is produced from a polymer sheet material as described herein.

The description of surgical heart valve 10 and collapsible/expandable prosthetic heart valve 100 are for context only. Thus, the polymer sheet materials described herein may be used in surgical heart valves that are similar to surgical heart valve 10 or surgical heart valves that are very different therefrom. Similarly, the presently disclosed polymer sheet materials may be used in collapsible/expandable prosthetic heart valves that are similar to prosthetic heart valve 100, or prosthetic heart valves that are very different therefrom, such as heart valves having a balloon-expandable stent; heart valves that do not have an aortic section; heart valves in which the stent has an hourglass profile, right cylindrical sections or ovoid cross-sections; heart valves with an end that flares outwardly; heart valves intended to replace other cardiac valves, such as the mitral valve; etc. For example, the stent may be made of a single or multiple bent wires such as illustrated in U.S. Pat. Nos. 5,411,552 or 5,855,601, forming a zigzag or sinusoidal shape, or may be made from interwoven or intercrossing bars such as shown in U.S. Pat. Nos. 5,545,214 and 7,585,321. The stent may also be formed of woven materials that can be such as shown in EP 2,926,766, which is hereby incorporated by reference herein for its teaching of a woven stent and for its teachings regarding the mounting of a cuff and/or sac on the interior or exterior of a stent. Often, however, the stent is made from a laser-cut nitinol tube. A balloon-expandable stent may be used and is often composed of biocompatible metals known in the art, including but not limited to, cobalt chromium, nitinol, and stainless steel. See U.S. Pat. Nos. 7,393,360; 7,530,253; 9,629,714, the disclosures of which are hereby incorporated by reference herein.

According to the present disclosure, so long as at least one component is made from a polymer sheet material as disclosed herein, other valve components, such as the inner and/or outer cuff(s) and/or one or more leaflets, and indeed components of other medical devices, may be made from a woven or knitted fabric, or from a felt or other polymeric fabric that is nonwoven.

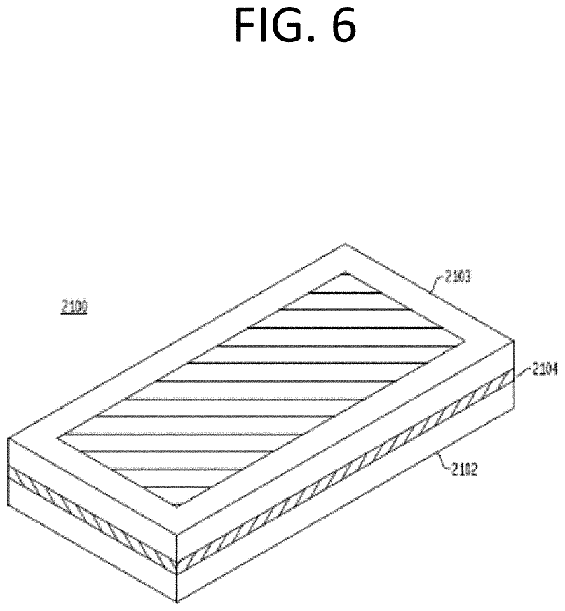

FIG. 5 is an exploded view of an exemplary multilayered polymer sheet 400 in accordance with the present disclosure useful for discussing its general structure in a non-limiting fashion. Sheet 400 can be created by heat laminating polymer layers 402 to a continuous or complete primary polymer film or ply 404. In FIG. 5, a single polymer film or ply 404 may be covered on only one surface by a single polymer layer 402, or may be sandwiched between polymer layers 402, one, two, or more on each side of the primary polymer film or ply (the latter is illustrated). Layers 402 may be applied as films or may be applied as coatings by techniques such as by dip coating, spray coating, 3-D printing and the like.

In some embodiments, up to about 40 total layers may be used to form a polymer sheet as disclosed. In the context of the embodiment illustrated in FIG. 5, 12 layers can be applied to one or to both sides of the primary polymer film or ply. In other embodiments, 1 to 10 layers of polymer may be applied to one or to both sides of the primary polymer film or ply. In still other embodiments, 1 to 5 layers of polymer may be applied to one or to both sides of the primary polymer film or ply. Thus, each side of the primary polymer film or ply can be covered, completely or partially, by 1, 2, 3, 4, 5, 6, 7, 8, 9, 10, 11, 12, 13, 14, 15, 16, 17, 18, 19, or 20 additional polymer layers.

When one or more applied layers is/are used to cover at least one major surface of the primary polymer film or ply, the various layers need not each have the same thickness or the same composition or orientation—they can all be different, not only different from the primary film or ply, but also different from one another. While even thicker polymer layers are possible for use and attachment to a primary polymer ply or film, generally speaking, the thickness of each of the polymer layers on each side, whether comprised of 1 layer or 20, may range from about 0.5 μm to about 100 μm, in another embodiment from about 0.5 μm to about 50 μm, and in some further embodiments from about 5 μm to about 40 μm. In one other embodiment, the thickness of the layers on each side may range from about 5 μm to about 30 μm. Very thin polymer layers, e.g., from about 0.50 μm to about 2 μm, may be applied simply to fill the open pores in the primary polymer film or ply or for other reasons. These applied layers may be films, plies or coatings.

If a partial layer is applied just at or adjacent an edge of the sheet, such as for example, the attachment edge of a leaflet, such that it can be sewn through when attaching the leaflet to a cuff and/or stent, it can be relatively thicker as it will not impact the flexibility of the balance of the leaflet. This is true regardless of whether this partial layer is applied to one or both major surfaces of a single continuous or complete layer or disposed between continuous or complete layers. Again, using a leaflet as an example of a component formed from a polymer sheet material in accordance with the disclosure, the thickness of the leaflet could vary along a gradient, such as from the attachment edge to the free edge of the leaflet.

It will be appreciated that the thicknesses of the polymer sheet materials described herein are dictated by a balancing of properties and functionality. The number and thickness of individual layers can have an impact on the size to which a collapsible medical device, such as a collapsible/expandable prosthetic heart valve, can be collapsed. For non-collapsible devices, such as surgical heart valves, collapsibility is not a factor dictating thickness. In such instances, other properties may dictate composition, number of layers and thickness, such as, without limitation, rigidity, porosity, stability and flexibility. For any device to be delivered in a minimally invasive manner, such as transluminally or laparoscopically, even though the device may not need to be collapsed or expanded, it still needs to have as small a diameter or cross-sectional area as possible. Of course, there are many other factors involved as well including, without limitation, the size and geometry of the stent or other medical devices to which the polymer sheet materials of the present disclosure may be applied or attached.

Leaflets, cuffs, or other structures of medical devices made with the sheet materials of this disclosure may be reinforced, weighted, shaped or have their flexibilities altered by the addition of other features, with or without coatings (that may also perform some of these same functions.) These other features may include, without limitation, structures such as sutures, wires and the like.

A suture could be attached in any number of ways and any number of locations. Using a valve leaflet for example, a single stitch could be placed in the middle of a major surface of the leaflet, or in a specific location on the free edge. A series or pattern of a plurality of individual stitches could be used as well. These could provide reinforcement, could alter the flexibility, or provide weight in order to, for example, bias the leaflet into a proper closed position. One or more sutures could be used to form one or a plurality of suture lines across an entire major surface or a portion or a major surface of a leaflet, along the attachment edge or along the free edge of the leaflet for any of the reasons just described. For example, a row of sutures could be added, of varying number of stitches, in a line or specified shape, across the full length of the leaflet in a single row from the attachment edge to the free edge of a leaflet. This row could be continuous or discontinuous row. The properties can be altered based on the number and density of stiches, the number of sutures applied and the location and pattern in which it (they) are applied. Using several such lines of sutures between the attachment edge and the free edge of a leaflet can alter the stiffness of the leaflet and create flexible zones or “hinges.” The use of these sutures could also help impart or preserve the shape in three dimensions of the leaflet or other structure.

One or more sutures could also be stitched to at least a portion of the attachment edge and/or the free edge of a leaflet to provide reinforcement and/or weight and or to introduce or preserve a shape. The use of sutures here could provide additional strength in an edge that could be sutured or otherwise attached to a cuff and/or stent and can help retard fraying or delamination at an edge. These suture lines could be formed of one or more sutures, can be continuous or discontinuous, and can either be extended in a narrow area or across the entire length of one of these edges.

It should be understood that leaflets and cuffs are often sutured to each other and/or to a stent. Frequently, as few sutures as required are used for this attachment, and in some instances a single suture could be used to attach each of the leaflets. Using a suture in this fashion, the suture is stitched a plurality of times—sometimes 10's or even 100's of times—through the leaflet and or cuff. Each of these is obviously a stitch. But, a “stitch” in the present context refers to a single stitch or knot of a suture material generally not used to attach any portion of the leaflet to a cuff, to another structure. A suture line in this instance is a suture stitched a plurality of times through a cuff or leaflet also not primarily intended to attach the cuff or leaflet to another structure. As used herein, a “continuous” suture line refers to a single suture line formed from a single continuous suture, or multiple sutures that are substantially continuous with one another (e.g. the first suture ends where the second suture beings). On the other hand, a “discontinuous” suture line refers to a suture line formed of two or more sutures, where the sutures are substantially discontinuous with one another (e.g. the second suture begins at a spaced location from where the first suture ends).

And in still another aspect of this embodiment, instead of, or in addition to coating, partial coatings, sutures or the like, other reinforcing structures such as wires, including without limitation, steel or nitinol wires, could be used. These structures could be inserted into the sheet along the attachment edge, the free edge or across some portion of the major surface of a leaflet, again used only for an example. They could also be applied by gluing, laminating, etc. or could be formed within a layer itself. They could be disposed between two adhered layers as well. The wire or other reinforcement may extend across the entire edge, just a portion of it, may be continuous or discontinuous. A “continuous” or “discontinuous” wire has a similar definition as the continuous or discontinuous suture line described above, except the reference material is a wire instead of a suture. A suture could be used instead of a wire, or other fiber which is attached by being glued or laminated between the sheet material and a layer or partial layer instead of being stitched. These features when added could also impart or preserve a three-dimensional shape to the sheet or the components made therefrom. For example, valve leaflets generally include a “belly” and assume a bowl or “spinnaker” like shape. A wire running through the apex or trough of the structure to maintain the curvature and shape.

In still a further embodiment, the medical device, or an element thereof, such as a sheet material leaflet, could be constructed or attached so as to form a pleat or fold across a major surface of the leaflet. In particular, this can be accomplished by suturing a gathering of the sheet material at the attachment edge and optionally by including structures, cuts or ablations on the face of the leaflet form folding zones or pleats.

Producing sheets of polymer materials useful in accordance with the present disclosure may be accomplished by any known method. U.S. Pat. No. 2,852,811, for example, describes methods for casting thin plastic films, particularly those composed of polytetrahaloethylene. As for reinforced polymer sheet materials, processes that can be used include those generally described in U.S. Pat. No. 4,610,918, which describes the production of fluoropolymer coated textiles, and U.S. Pat. No. 7,109,135, which relates to a woven fabric sandwiched between PTFE layers. These teachings, however, must be modified to include reinforcing materials instead of a woven fabric.

The manufacturing methods also include the step of layering a first or primary layer of polymer material with one or more additional layer(s) of polymer material to form a polymer sheet. This may be done by stacking layers with shapes that are already cut or formed into the desired shape or by, for example, stacking sheets and then cutting a structure therefrom. These layers are affixed to each other by any known process, for example, gluing, reacting, crosslinking, folding, sewing, riveting, spot welding or laminating. One adhesive that can be used for UHMWPE is Reltek's BONDIT B-45′. Coatings may be formed in situ by spray coating or dip coating, vapor depositing, powder coating, electro plating, 3D printing or the like on one or more side edge or major surface of the at least one continuous or complete polymer layer.

Lamination is one of the most common methods to join layers and can be achieved through several mechanisms as is known in the art, including heat lamination and solvent lamination. The preferred method used to laminate sheets together is through a heat lamination process. In addition to applying heat, it is often desirable to apply pressure during lamination, during heat treatment or after heat treatment, such as through the use of heated rollers. Lamination processes may occur at a temperature of from about 100 to about 350 degrees centigrade depending on the exact process and the polymers being used. In other embodiments, the temperature of the lamination process may be from about 200 to about 280 degrees Centigrade. These temperatures are based on standard pressure—the temperature range can vary when higher pressure is used. For urethane materials, the lamination temperature generally is between about 120 and about 280 degrees Centigrade.

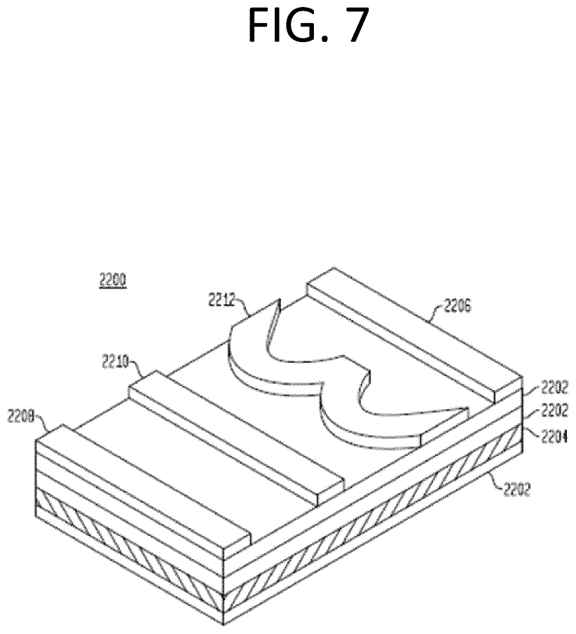

FIGS. 6-8 and 17-25 illustrate certain exemplary structures that can result from the production of polymer sheet materials with or without partial layers. For a partial layer, polymer films or plies of the desired shape and size can be formed or cut from larger films or plies and placed where desired on a primary layer and glued, laminated, etc. in place. Liquid polymer can be sprayed on or otherwise applied to a primary layer by dipping, depositing, etc. to form a partial layer thereon. A polymer can be molded separately to form a partial layer and applied to a primary layer or can be molded to the shape desired directly on a major surface of the primary layer. Liquid materials, particles, slurries, etc. could also be applied by 3D printing (indeed, complete or continuous layers could be made using 3D printing, which also can be used to contour or pattern a surface.) A partial layer may also be achieved by applying a film or coating as a layer over all or most of a major surface of the primary polymer layer and then removing unwanted portions of that applied layer by ablating, melting, evaporating, cutting, eroding or frictional removing (sanding, grinding, rubbing). Thus ribs, reinforced areas adjacent an attachment region, and structures at or adjacent the free edge used to resist wear of a leaflet can all be formed by removing material from an applied layer.

Ablation can also be used to provide a pattern in a layer or to impart other surface features. Ablation could be used, for example, to taper the thickness of a leaflet, just for example, from an attachment edge to the free edge. This may be accomplished by progressively ablating the topmost or bottom most layers from one edge to the other, deeper and deeper, thus removing more and more material. As another example, ablation could be used to remove a portion of the layers in a selected area, such as in the portion of a leaflet that will form its belly when in use in a heart valve, to provide additional flexibility to that region. Other surface patterns may also be developed. In addition, surface roughening, such as to promote cell adhesion generally or in specific areas of the surface, may be employed.

When ablation is used, the topmost layers that will be selectively ablated could be composed of one polymer material, with one or more under-layers that are not to be removed or patterned being composed of a different polymer material. See, for example, FIG. 20, which contains a primary polymer layer 3751 disposed between a first polymer layer 3740 and a partial layer feature 3750 adjacent the free edge 3730. Partial layer 3750 could be obtained by cutting out from a film and applying it to polymer layer 3571 or it could be formed by ablating away a portion of a top layer, leaving only exposed portions of layer 3750. A further partial layer 3725 is located on the opposite side and can be formed in the same or in a completely different way. The edge structure 3751 could be formed by dip coating and then ablating away any excess, and layer 3725 could be cut from a separate polymer film and applied and attached.

Thus, a polymer layer or layers may form a pattern or relief on one or both sides of a complete or continuous polymer layer. The pattern or relief that is formed may vary in thickness; provide rigidity or additional cohesion to specific regions; retard fraying; reinforce shape, stretch, or friction; alter porosity; provide or encourage cell attachment or prohibit it in specific areas; enhance coaptation; and the like.

FIG. 6 illustrates a non-limiting example of a multilayered sheet material 2100 which may be patterned as shown. A primary polymer layer 2104 is covered with a polymer layer 2103 in only the areas a few millimeters from each edge of the top major surface of the primary polymer layer 2104—forming a structure looking like a picture in a frame. Layer 2103 could be applied as a full film, and the central portion ablated. A partial layer could be formed by cutting out the center of a film or ply and applying the remainder adjacent the side edges of primary polymer layer 2104. Or, a coating could be applied on the top major surface adjacent the side edges and allowed to dry/cure and harden, thereby forming layer 2103. Layer 2103 could also be 3D printed around the periphery of primary layer 2104.

The bottom major surface of primary polymer layer 2104 may be continuously coated with a polymer or other material to form continuous layer 2102 or a full-sized coextensive film or ply could be applied to form continuous layer 2102. A checkerboard pattern, a series of strips, concentric circles or other shapes may be laminated, printed, etched, masked, coated or otherwise formed onto one or more major surfaces in the same overall fashion. Each of these patterns can be formed using differing thicknesses and/or different numbers of layers.

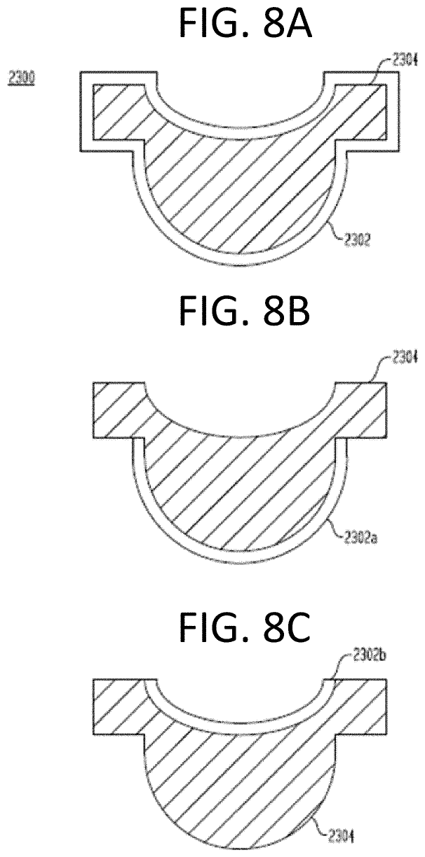

FIG. 7 illustrates another example of a partially layered polymeric sheet material 2200 in which a first continuous or complete polymer layer 2204 is laminated on its entire bottom major surface with a second continuous or complete polymer layer 2202. Two additional polymer layers 2202 are also applied and attached to the upper surface of polymer layer 2204. An additional polymer layer may be 3D printed or otherwise applied in a discontinuous fashion over the continuous layers. (See 2206, 2208, 2210 and 2212.) These features may also result from ablation or otherwise, as already discussed. A portion of this additional layer may be applied to the topmost continuous layer 2202 so as to overlie or be adjacent two opposed edges 2206 and 2208 of the continuous layer. This could be done to reinforce those areas which may be involved in attaching this structure to a medical device, such as by using sutures to sew it to the luminal and abluminal surfaces of a stent such that it is wrapped around the inflow end of the stent to provide internal and external cuffs. Another portion of the additional layer may include a curved portion 2212 to help reinforce that portion of the resulting inner cuff to which leaflets could be sutured. The additional layer may also include another strip 2210 located in the area that will actually wrap around the inflow end of the stent to help prevent abrasion upon contact between the cuffs and the stent and to provide a sturdier portion for suturing to the inflow end of the stent.

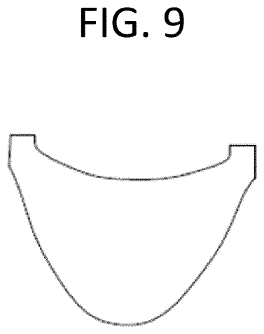

FIGS. 8A-8C, for example, illustrate a patterned polymer sheet for use in a leaflet 2300. The primary polymer layer 2304 may be discontinuously coated with a polymer layer 2302 such that only the area a few millimeters from each edge of the sheet is coated. On the other hand, a continuous layer could be applied to the primary layer and ablated, or a thin polymer strip could be attached or 3D printed in only discrete surfaces of primary polymer layer 2304 so as to create the multilayered structures illustrated. The pattern as just described may be used for a leaflet in which only the attachment edge and the free edge of the leaflet are coated, as shown in FIG. 8A. In some embodiments, the pattern as just described for a leaflet may be created by applying an additional polymer layer discontinuously, creating an area extending about 10 mm from the attachment edge and the free edge that is covered with the additional polymer. In other embodiments, the covered areas may not be uniform and the primary polymer layer 2304 may be covered in an area extending about 10 mm from the attachment edge with polymer 2302a and in an area extending about 5 mm from the free edge with polymer 2302b, as is shown in FIG. 8B and FIG. 8C, respectively. FIG. 8B illustrates the underside (or upstream surface) of the leaflet which attaches to the stent, while FIG. 8C illustrates the other side (or downstream surface) of the leaflet. The reverse may also be possible when used for a leaflet.

The polymer sheet materials of the present disclosure may include large holes or structures or patterns for various functions. Those used to facilitate suturing or attachment will be described elsewhere. However, other structures are possible. Indeed, a mesh can be formed from the sheet material by cutting holes or openings through the entire thickness of the sheet in a regular pattern. A mesh could also be created by making several perforations and then stretching the material such that the opposed edges of the perforations are drawn apart, forming openings. Assume that a cuff material is going to extend up the inside or outside of a stent of an expandable aortic heart valve to a level where, when implanted, there is a concern that it could impede access to the coronary arteries. A hemispheric notch or notches could be cut in the downstream edge to avoid that issue. Or if the cuff extends up into the aorta, large holes could be cut in the side to accommodate access to the coronary arteries. A graft used in the abdominal aorta could have similar holes intended to accommodate the openings to the renal arteries. These holes or structures could be formed as part of each sheet or cut into them after a single or multilayered sheet material has been formed.

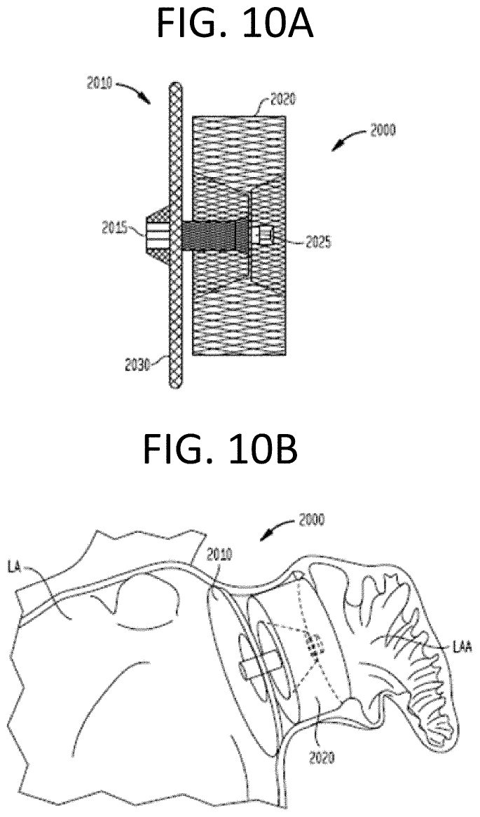

FIG. 9 depicts a heart valve leaflet fabricated from a sheet material composed of UHMWPE layer(s). The sheet material may be cut to a desired geometry by stamping, mechanical cutting, laser cutting or other known techniques. Laser cutting may melt the edges of the leaflets to effectively create a single, continuous seam which may be particularly useful for retarding delamination if the polymer sheet material is composed of multiple layers. The leaflet could also be molded by pouring liquid material into a mold and letting it set or by 3D printing.

Additionally, there may be a preference for a smooth transition between the main leaflet body and the edges of the leaflet. If the transition is not smooth, blood cells may encounter a relatively large amount of shear stress at the transition point, which can activate the blood cells, creating a potential for undesirable thrombus formation. The edges of the leaflet may be coated with a polymer or other material as described above to ensure a smooth transition between the main leaflet body and the edges of the leaflet.

In still further embodiments, at least one of the polymer sheet materials produced may be reinforced with reinforcing fibers or other structures. Leaflets, for example, may be composed of such a reinforced polymer sheet material and fabricated or cut such that its fibers are at a bias angle of between about 30 degrees and about 60 degrees relative to a line that extends perpendicular to the free edge of the leaflet when the leaflet is in a flattened condition or lies within a plane (e.g., before the leaflet is attached to the valve assembly). In another embodiment, some or all of the leaflets used in a device may be fabricated with their fibers at that same relative bias or at different relative biases. Thus, the leaflets in a given device may not all be fabricated with their fibers at the same bias. In one such embodiment, all of the leaflets may be fabricated with their fibers on a bias of between about 30 degrees and about 60 degrees relative to a line that extends perpendicular to the free edge of the leaflet, but the fibers of at least one of the leaflets may not be on the same bias as the fibers of the other leaflets of the valve assembly. In another such embodiment, the fibers of at least one leaflet may be biased at between about 30 degrees and about 60 degrees relative to a line that extends perpendicular to the free edge of the leaflet and the fibers of at least one other leaflet may not be. The same sort of biasing can apply to the directional orientation of reinforcing fibers incorporated in one or more polymer sheet materials used for other prosthetic valve components or other medical devices.

In some embodiments, the polymer sheet materials may be produced using layers that are directionally oriented in the same or in different directions. Directionally oriented layers are generally composed of polymer films that are formed or stretched in a particular direction that can alter their structure and/or properties. In attaching components to a stent and/or to another supporting structure of a medical device, the polymer sheet material may be attached such that the polymer sheets are oriented in a particular direction. These polymer sheet materials may be cut or formed and attached to the support structure such that the direction of at least one of these orientations is substantially parallel or perpendicular to the longitudinal axis of the support structure. Where several polymer layers are laminated or attached to one another to form a multilayered sheet, one layer could be oriented generally perpendicular to the longitudinal axis of the support structure and the other layer could be oriented generally parallel to the longitudinal axis. Alternatively, these sheet materials and structures made from them may be mounted to the support structure such that the directional orientation is generally on a bias, i.e., at an oblique angle, to the longitudinal axis of the support structure. In one example, a first layer may be oriented in a first direction and a second layer may be oriented in a second direction that is perpendicular to the first direction. A third polymer film may be oriented in a third direction that is about 45 degrees between the first direction and the second direction. Such directionally oriented sheet materials may, for example, be used to form an inner cuff and/or an outer cuff of a collapsible/expandable heart valve or the skirt of a surgical heart valve. When used for an inner cuff or an outer cuff of a collapsible/expandable heart valve, the oblique angle may be between about 30 degrees and about 60 degrees relative to the longitudinal axis of the support structure when the heart valve is in an expanded use condition. In some embodiments, the sheet material may be oriented such that the directional orientation is at about 45 degrees relative to the longitudinal axis of the support structure when the heart valve is in an expanded use condition. (See EP 2,949,292, the disclosure of which is hereby incorporated by reference herein, for its teaching of the manufacture and attachment of a cuff or leaflet so that the direction of orientation of the material forming the cuff or leaflet is at an oblique angle relative to the longitudinal axis of a stent.)

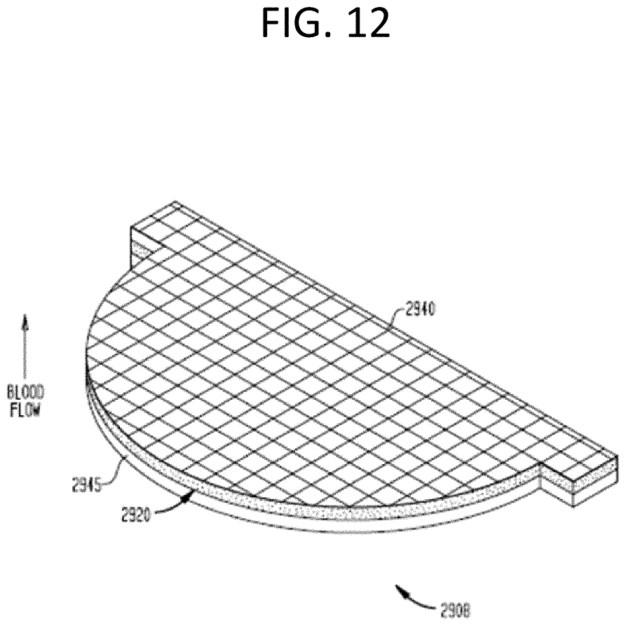

FIGS. 11-29A further illustrate the structural diversity of leaflets used in medical devices in accordance with the present disclosure, such as collapsible, expandable and surgical heart valves. It should be understood, however, that these structures are illustrative and that the polymer sheet materials depicted can be used in other medical devices and their shape, thickness, and composition may be adjusted 4to suit that particular purpose.

FIG. 11 illustrates leaflet 2808, formed from a complete or continuous layer 2840 of polymer material such as, without limitation, UHMWPE. Leaflet 2808 has a first major surface or downstream surface 2815 and a second major surface or upstream surface 2820. Leaflet 2808 is similar to leaflet 108 shown in FIG. 2 and is attached to a stent so as to form a one-way valve assembly. The actual surface illustrated in FIG. 2 is the first major surface or downstream surface 2815 as blood flows into the valve from the inflow or annulus end 130 to the outflow or aortic end 132. As used herein, the first major surface is considered the downstream side with the downstream surface 2815 and the opposite major surface is the upstream side with the upstream surface 2820. Stated in another way, the downstream surface 2815 is the major surface generally facing the outflow or aortic end 132 of the stent when the valve leaflets are in a closed position during use. The upstream surface 2820 generally faces the inflow or annulus end 130 of the stent when the leaflets are in the closed position.

Leaflet 2808 has a free edge 2830, an attachment edge 2825, and a plurality of tabs or flaps 2835. Generally, the leaflet is attached to the cuff and/or to the stent at or adjacent the attachment edge 2825. The tabs 2835 often form commissures at which two adjacent leaflets meet. Each tab 2835 is often attached to an adjacent tab of an adjacent leaflet and/or to the stent at, for example, a commissure attachment feature such as element 116 in FIG. 2. While much of the leaflet moves during operation of the prosthetic heart valve, the greatest degree of movement is experienced by the free edge 2830. It is pushed out of the way from the center of the valve toward the luminal surface of the stent when blood is flowing, and is pushed back toward the center of the valve where it engages or coapts with other leaflets when the valve is closed.

As noted, the leaflet 2808 in FIG. 11 is uncoated, and not a laminate or multilayered structure. Leaflet 2808 is illustrated as a single layer of polymer sheet material, although multiple layers could be stacked directly atop one another and attached to one another by suitable methods.

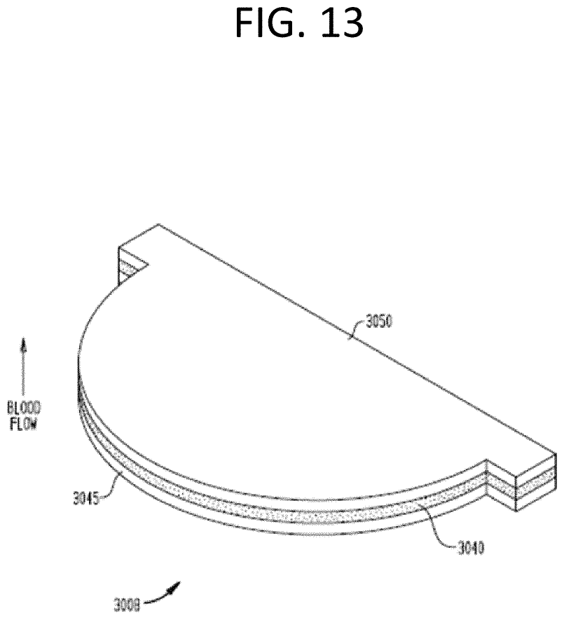

FIG. 12 illustrates a leaflet 2908 produced from a multilayered polymer sheet material and is generally of the same structure and composition as that illustrated in FIG. 11, other than it includes a plurality of layers. The leaflet 2908 in FIG. 12 includes a first polymer layer 2940, which can be composed of any of the polymers disclosed herein, as well as a second polymer layer 2945. Polymer layer 2945 is generally coextensive with the shape and size of polymer layer 2940, and the layers are attached to one another by lamination, gluing, or other known methods. Polymer layer 2940 and polymer layer 2945 are illustrated as being of roughly the same thickness, however, that need not be the case. Moreover, the polymers used for the two layers may be the same or different.

FIG. 13 illustrates a valve leaflet 3008 that is generally similar to those shown in FIGS. 11 and 12 comprised of a first polymer layer 3040, a second polymer layer 3045 covering the entire upstream surface of the first layer and a third polymer layer 3050 covering the entire downstream surface of the first polymer layer. As before, the individual layers can be made of any of the polymers described herein, and the polymers used for the three layers may all be the same, may all be different, or two layers may be formed from a different polymer than the third layer. While a leaflet having three layers is illustrated, more layers are possible, and the layers may have different thicknesses.

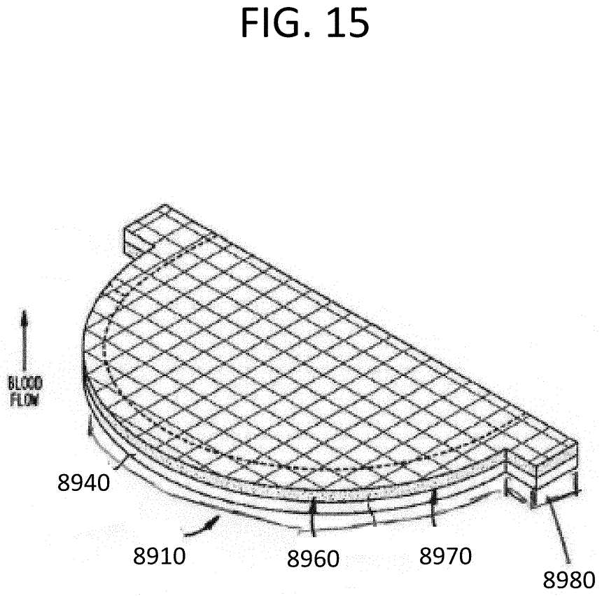

Similarly, FIG. 14 illustrates a leaflet 3108 that is similar to those shown in FIGS. 11-13. Leaflet 3108 has a multilayered structure in which the first or primary polymer layer 3140 is covered on both of its major surfaces with at least one additional polymer layer. A single polymer layer 3145 covers the entire upstream surface of primary layer 3140. There are, however, three polymer layers covering and attached to, directly or indirectly, the entire downstream surface of primary layer 3140. The most downstream or outermost layer 3150 may be made of ultra-high molecular weight polyethylene (UHMWPE), the next adjacent layer 3151 may be made of low density polyethylene, and the third and final layer 3152 situated against the primary layer 3140 may also be composed of UHMWPE.

As illustrated in FIG. 14, the three polymer layers 3150, 3151, and 3152 may have roughly the same combined thickness as polymer layer 3145 disposed on the upstream surface of primary layer 3140. This need not be the case. Each of polymer layers 3150, 3151, and 3152 may be thin or thick and their combination may be thicker or thinner than polymer layers 3140 or 3145. Moreover, while three polymer layers are illustrated on the downstream surface of primary layer 3140, as discussed elsewhere herein, the number of layers that can be applied to any one major surface of the primary polymer layer can be as many as 20 layers.

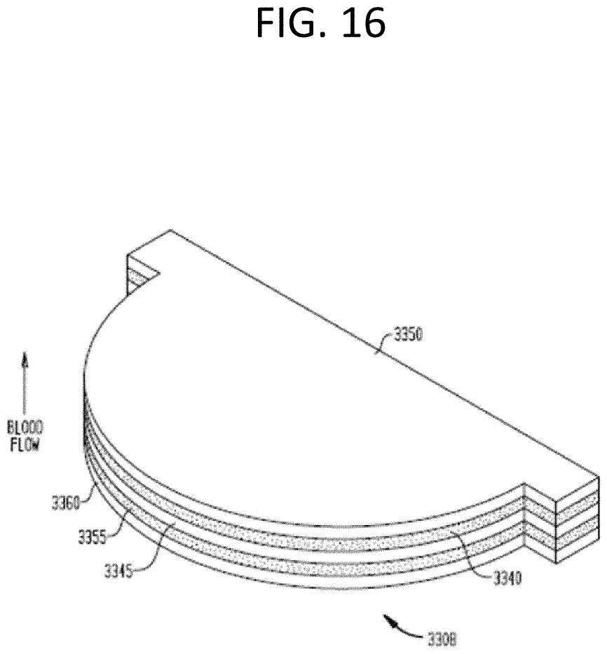

FIG. 15 is a schematic perspective view of a leaflet 8910 formed from a multilayered complete or continuous polymer sheet material according to the present disclosure comprising a first continuous layer 8960 and a second continuous layer 8980 directly affixed together over most of their respective facing major surfaces. However, there is a relatively thin curved partial layer 8970 affixed to and disposed between the continuous layers 8960 and 8980 at or adjacent their attachment edges, thus forming the attachment edge 8940 of leaflet 8910. This creates a relative thickening and reinforcement of the leaflet in the region along which it will be connected to a cuff and/or stent of an expandable heart valve. This edge may also include holes or grommets as shown and described in connection with later embodiments. Such holes or grommets may be disposed only in the partial layer or throughout the entire thickness of the construction.

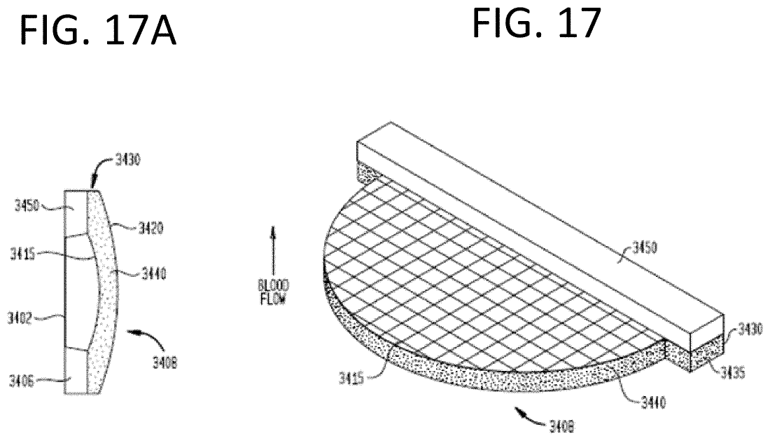

FIG. 16 illustrates yet another possible construction of a leaflet that is similar to those described above. Leaflet 3308 is constructed with three continuous or complete polymer layers 3340, 3345 and 3355. Additionally, the downstream surface of polymer layer 3340 is coated to form polymer layer 3350 and the upstream surface of polymer layer 3355 of leaflet 3308 is covered with a film to form polymer layer 3360. Each of the polymer layers can be of the same or different thicknesses, can be made of the same or different materials, can be have their polymers oriented in the same direction or in different directions, and can be reinforced or not. Additionally, while the foregoing describes layer 3350 as being formed by coating and layer 3360 being formed by a film, either or both of the layers may be formed by coating or a film.

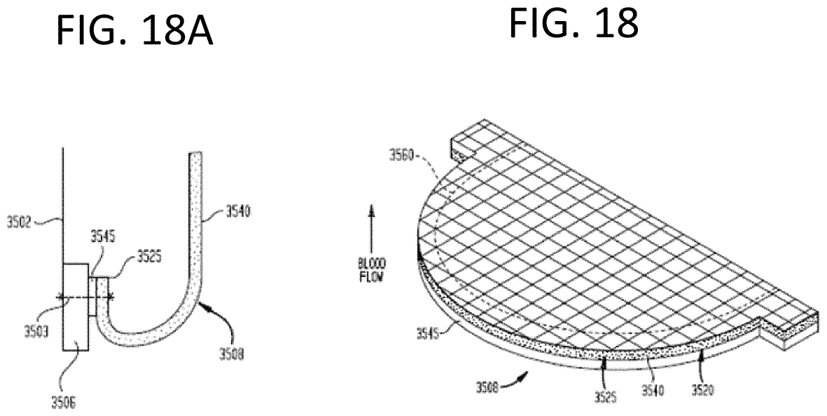

FIG. 17 illustrates a partially covered leaflet 3408. Leaflet 3408 comprises a complete or continuous polymer layer 3440 and also includes a partial layer 3450 disposed on its downstream surface 3415 adjacent the free edge 3430 of the leaflet. Partial layer 3450 may be formed by applying a coating or film to the downstream surface of polymer layer 3440. This partial layer 3450 is illustrated as being the same width as the leaflet, including tabs 3435, and roughly the same thickness as polymer layer 3440. However, that need not be the case. Partial layer 3450 may be wider or narrower across the downstream surface 3415 of polymer layer 3440 and may be thicker or thinner than the full polymer layer. That said, partial layer 3450 is often thinner than and not as wide as the complete polymer layer. Polymer layer 3440 may include multiple continuous or complete polymer layers as opposed to the single layer illustrated. Multiple partial layers may also be used. The partial layer 3450 adjacent the free edge 3430 of leaflet 3408 may serve one or more purposes. For example, it may help add weight to bias the leaflet back into a closed position, it may help the leaflet retain its intended shape, and may promote or prevent cell attachment and proliferation adjacent the free edge 3430.

FIG. 17A is a partial cross-section of a stent and a valve assembly similar to those shown in FIG. 2. A portion of the stent 3402 is illustrated in cross-section with an internal cuff 3406 attached to a luminal surface of the stent. Leaflet 3408 is attached to cuff 3406 and/or stent 3402 at or adjacent its attachment edge 3425, which may be sutured to the cuff and/or stent. Leaflet 3408 is illustrated in its open position as it extends generally downstream to accommodate blood flow from the inflow end of the stent to the outflow end past the upstream surface 3420 of the leaflet. Partial layer 3450 is disposed on the downstream surface 3415 of the leaflet adjacent the free edge 3430 and is illustrated engaging the luminal surface of stent 3402. Partial layer 3450 therefore prevents direct contact of the polymer layer 3440 with the inner surface of the stent during blood flow, thereby providing additional wear resistance and helping to prevent the fraying of the free edge 3430. In addition to providing resistance to wear, such partial layer 3450 could help maintain the shape of the leaflet and its ability to coapt with other leaflets. Without partial layer 3450, inter-cellular attachment could exert forces that could tend to pull the free edge out of proper position. Instead of, or in addition to, a partial layer 3450 on the downstream surface 3415 of leaflet 3408, a similar partial layer may be applied to the upstream surface 3420 of the leaflet, adjacent the free edge 3430 or otherwise, to resist the deformation of the leaflet due to cellular ingrowth.

Partial layer 3450 is shown extending fully across the entirety of the free edge 3430 of leaflet 3408 between tabs 3435. This need not be the case. Partial layer 3450 may be provided adjacent free edge 3430 but not overlying tabs 3435. Further, partial layer 3450 may be a discontinuous layer of two, three, or more films or coated portions forming, in essence, a dashed line adjacent free edge 3430. Still further, partial layer 3450 may be formed of spots or dots applied intermittently adjacent free edge 3430. Each dot or each dash may have a different thickness and/or may be composed of a different polymer composition.

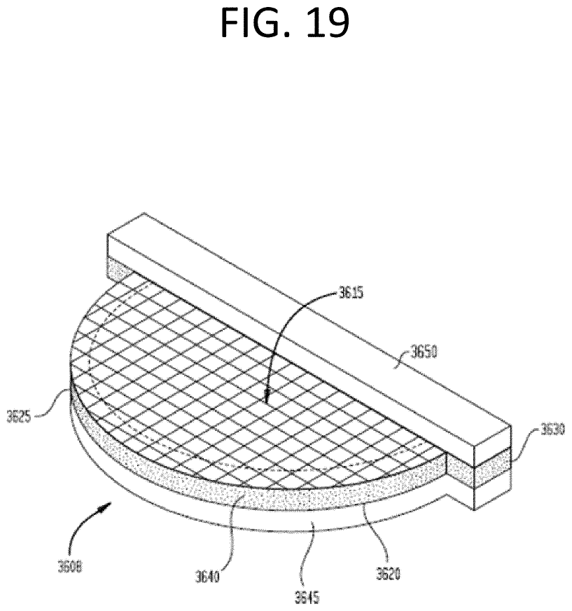

FIG. 18 illustrates another embodiment of a leaflet that is generally similar to those described above. Leaflet 3508 includes a primary polymer layer 3540 and a partial layer 3545 disposed on and covering a portion of the upstream surface 3520 of the primary layer. Indeed, partial layer 3545 does not cover the entirety of the upstream surface 3520 of polymer layer 3540. It is a relatively narrower strip in width and runs adjacent the attachment edge 3525, extending inwardly therefrom for some predefined width. An illustrative width is shown using the dashed semicircular line 3560 in FIG. 18. Partial layer 3545 may be formed by applying a coating or film to the upstream surface 3520 of primary layer 3540. FIG. 18A is a partial cross-section of a stent 3502 and a valve assembly similar to those illustrated in FIG. 2. Attached to a luminal surface of stent 3502 is a cuff 3506. Leaflet 3508 as shown is composed of complete polymer layer 3540, which is rolled or folded adjacent its attachment edge 3525 for attachment purposes. Disposed between layer 3540 and cuff 3506 is the partial layer 3545, which is provided adjacent the attachment edge 3525 of polymer layer 3540. As is true for FIG. 17A, leaflet 3508 is illustrated in the open position, e.g., a position similar to that which could result when blood is flowing through the valve from the inflow end of the stent to the outflow end. Leaflet 3508 may be attached via a suture 3503 anchoring both polymer layer 3540 and partial layer 3545 to cuff 3506 and/or stent 3502.

Partial layer 3545 need not be a single layer nor need it be the same thickness or composition as polymer layer 3540. As was previously described, its width need not extend over the entire upstream surface 3520 of polymer layer 3540. Indeed, generally, it may be provided with sufficient width to allow only a suture therethrough. Partial layer 3545 may provide additional reinforcement and/or may help prevent fraying when suturing leaflet 3508 to cuff 3506 and/or stent 3502. It may serve other purposes as well.

Partial layer 3545 is illustrated as being disposed on the upstream surface 3520 of polymer layer 3540. However, it may be disposed on the downstream surface or on both the upstream and downstream surfaces to provide additional reinforcement and/or other advantages. Partial layer 3545 also is illustrated as covering the entire attachment edge and tabs of leaflet 3508. That need not be the case. It need not be provided at the tabs and/or may be provided as discontinuous dashes or spots of varying compositions, numbers of layers and thicknesses as previously described in connection partial layer 3450 in FIG. 17.

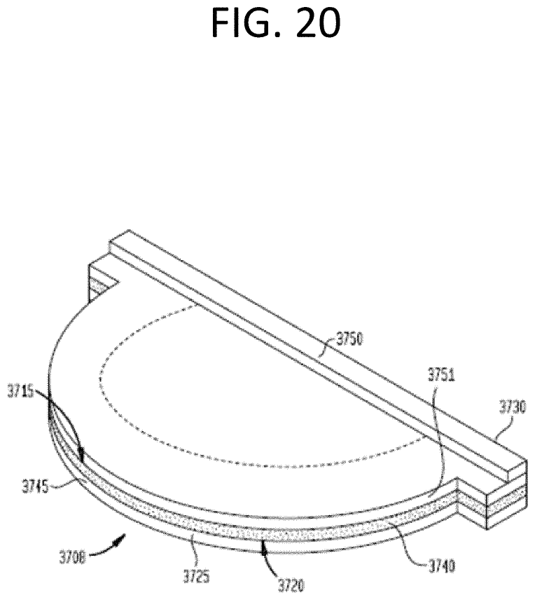

FIG. 19 is an amalgam of the leaflets illustrated previously in FIGS. 17 and 18. It includes a primary polymer layer 3640 having a partial layer 3650 attached to its downstream surface 3615 adjacent its free edge 3630. It also includes a partial layer 3645 attached to the upstream surface 3620 of polymer layer 3640 adjacent the attachment edge 3625. Partial layers 3645 and 3650 may each be formed by applying a film or coating to primary polymer layer 3640, or one partial layer may be formed by applying a film, and the other may be formed by applying a coating.

FIG. 20 illustrates another embodiment of a leaflet 3708 that is similar to those described above. Leaflet 3708 includes a primary polymer layer 3740 having another polymer layer 3751 applied to its entire downstream surface 3715. Adjacent the free edge 3730 of the leaflet is a partial layer 3750 applied atop/upon polymer layer 3751. Partial layer 3750 may be applied as a film or a coating as previously described, such as, for example, partial layer 3450 in FIG. 17. Leaflet 3708 also includes a partial layer 3745 attached to the upstream surface 3720 of primary polymer layer 3740 adjacent the attachment edge 3725, generally as described for partial layer 3545 in FIG. 18.

As illustrated in FIG. 20, however, the width of partial layer 3745 adjacent the attachment edge 3725 of leaflet 3708 is much greater than the width of partial layer 3750 adjacent the free edge 3730 of the leaflet. This is meant merely to illustrate the fact that there are partial layers on various surfaces of a leaflet and that they may independently cover different widths or areas. The same or different materials may be used for the various layers.

Note also that in the foregoing illustrations, partial layers are generally shown as single structures. However, partial layers, whether films or coatings, could be stacked atop one another and could have the same area or successive partial layers could have progressively smaller areas, thus forming a pyramidal sort of structure. Further, partial layers, whether formed by films or coatings, can themselves be coated with one or more coatings—partial or as part of a fully coated structure. Each partial layer may have the same or a different composition and the same or a different thickness as well.

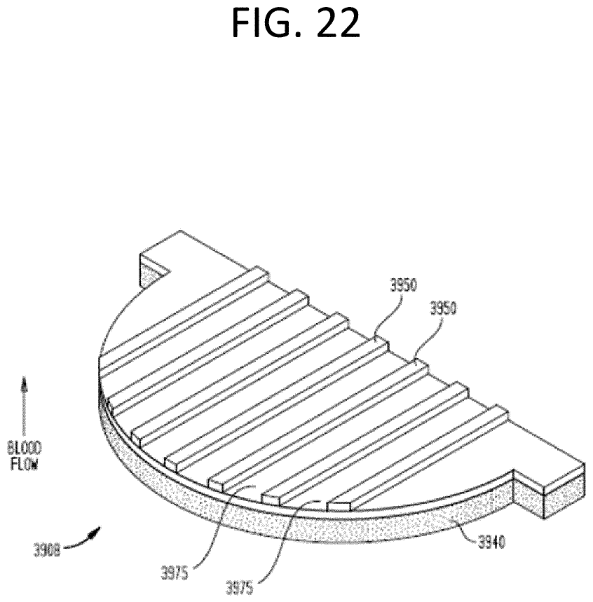

FIG. 21 illustrates another embodiment of a leaflet 3808 that is similar to those described previously. Leaflet 3808 contains a primary polymer layer 3840 similar to the primary polymer layers previously described. Disposed on the downstream surface 3815 of the leaflet are one or more “ribs” or reinforcing strips 3850 composed of one or more partial layers. These ribs are shown as running from approximately the attachment edge 3825 to the free edge 3830 of leaflet 3808. Reinforcing ribs 3850 may provide weight and structure to bias the leaflet from an open position back to a closed position. They may also provide some measure of structural rigidity and reinforcement to leaflet 3808. While shown as extending from the attachment edge 3825 of the leaflet to the free edge 3830, that may not be the case. Ribs 3850 may extend from attachment edge 3825 approximately halfway along the downstream surface 3815 of the leaflet toward the free edge 3830. Similarly, they may extend from adjacent free edge 3830 approximately 30% of the way along the downstream surface of primary polymer layer 3840 toward the attachment edge 3825. Ribs 3850 may be of any length, thickness, width, number of polymer layers and composition.

While ribs 3850 are shown applied to the downstream surface 3815 of primary polymer layer 3840, they could be applied to the upstream surface 3820 thereof instead of, or in addition to, their application to the downstream surface. Moreover, an additional polymer coating (not shown) may be applied to the entire downstream surface of leaflet 3808 to provide a smooth, if undulating, surface topography. A similar polymer coating could be applied to the upstream surface 3820 of the leaflet if ribs 3850 were applied to the upstream surface.

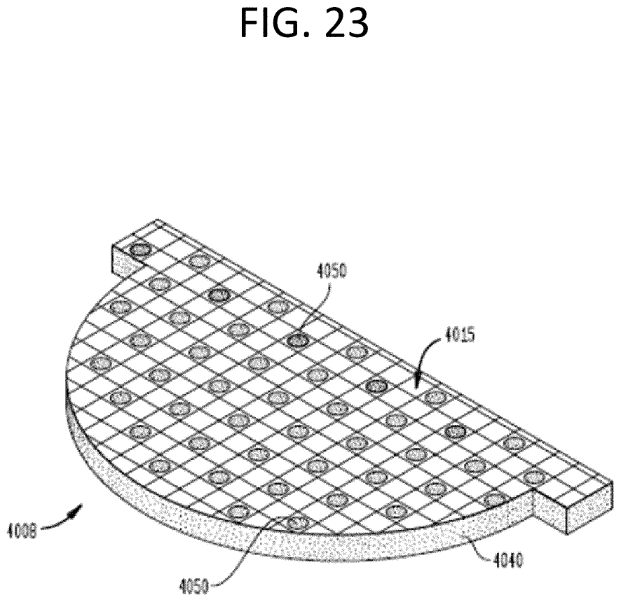

This concept of reinforcing ribs is further illustrated in FIG. 22 in which leaflet 3908 contains a plurality of ribs 3950 again extending from adjacent the attachment edge to the free edge of primary polymer layer 3940. In addition to providing reinforcement, shape and biasing as previously described in connection with the leaflet in FIG. 21, the spaces 3975 between ribs 3950 may act as folding regions helping to provide a controlled fold of the leaflet when the prosthetic heart valve is collapsed for loading into a catheter for transcatheter or transapical delivery.

In a variant hereof, leaflet 3908, or any leaflet described herein, may be scored on its upstream surface or downstream surface, such as by laser ablation, to produce a pattern on the surface. Such pattern may facilitate folding of the leaflet during collapsing of the prosthetic heart valve, may increase the flexibility of the leaflet for opening and closing during use, or may improve the performance of the leaflet in other ways.

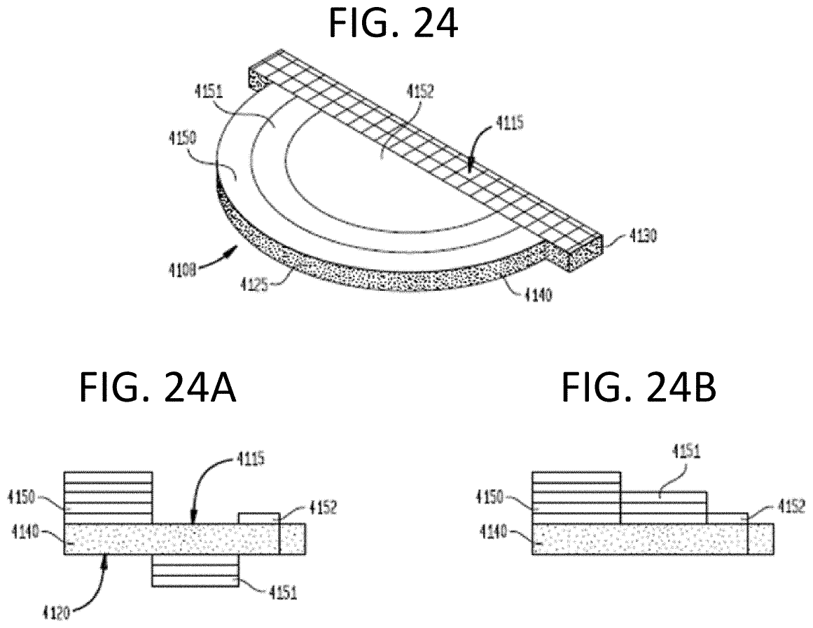

FIG. 23 illustrates another leaflet embodiment. Here, leaflet 4008 comprises a primary polymer layer 4040 having a downstream surface 4015 to which are attached one or more polymer dots or spots 4050. Like the polymer ribs illustrated in FIGS. 21 and 22, the spots 4050 may be considered partial layers formed by applying a film or coating to primary layer 4040 and can provide weight to help bias the leaflet to a closed position during use. Spots 4050 may also provide selective reinforcement and/or abrasion resistance. While shown as spots or dots in FIG. 23, these spots could be present in any number and in any shape such as, without limitation, crosses, lines, dashes, polygons, etc.

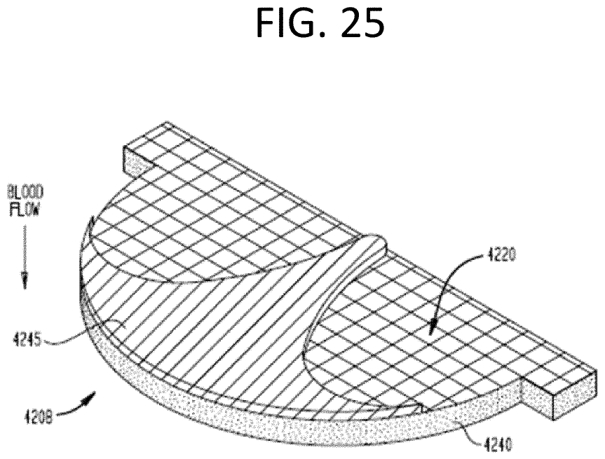

FIGS. 24, 24A, and 24B illustrate other partial layering arrangements for a leaflet—although, as with all of the leaflet figures described herein, the techniques and arrangements of layers depicted can be applied to any polymer sheet to be used in any medical device or component thereof. As with the other partial layers described herein, the partial layers of FIGS. 24, 24A and 24B may be formed by applying one or more films, coatings or a combination of films and coatings to a primary polymer layer. In FIG. 24, partial layers are disposed on the downstream major surface 4115 of the primary polymer layer 4140 of leaflet 4108. A first semicircular partial layer 4150 of a predetermined width may be comprised of five individual polymer layers that may be the same as or different from one another in composition and thickness. Disposed relatively inwardly from the stack of layers 4150, toward the free edge 4130 of leaflet 4108, is a second concentric semicircular partial polymer layer 4151 comprised of three different polymer layers. These layers may have the same compositions and thicknesses or different compositions and/or thicknesses from those used in the layers of partial layer 4150. They may have the same or a different width as well. Finally, further inwardly and closer to free edge 4130 is partial layer 4152 composed of a single polymer layer. Partial layer 4152 may be made of one of the polymers used in partial layers 4150 or 4151, or may be made of a different material altogether. It may have a width that is the same as or different from those of partial layers 4150 and 4151. The area directly adjacent free edge 4130 in this embodiment is uncovered. This entire structure could be covered with an additional film or coating that would provide a smoother surface, albeit one gradually getting thinner from attached edge 4125 to free edge 4130.

FIG. 24A shows a similar construction, however, partial layer 4151 is disposed on the upstream surface 4120 of the leaflet as opposed to the downstream surface 4115. Partial layer 4150, composed of five individual polymer layers, and partial layer 4152, composed of a single polymer layer, are disposed on the downstream surface 4115.

FIG. 24B shows a similar construction, however, instead of being semicircular or forming a rainbow, the partial layers are formed as parallel strips, with the first strip 4150 running roughly parallel to the free edge 4130 composed of five individual polymer layers, the next strip 4151 composed of three polymer layers and the last area 4152 composed of a single polymer layer.

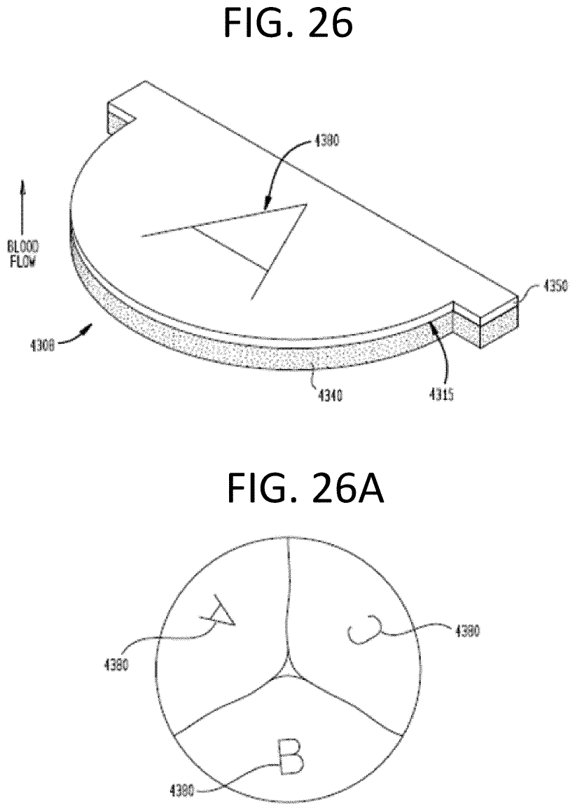

FIG. 25 illustrates a partial layer applied to the upstream surface of a primary polymer layer to form a leaflet 4208. In particular, leaflet 4208 is shown comprising a primary polymer layer 4240 and applied to its upstream surface 4220 is a reinforcing partial layer 4245. Partial layer 4245 may be formed by applying one or more films or coatings to primary layer 4240, forming at least one polymer layer, and possibly a plurality of polymer layers, in any shape or size as described in connection with FIGS. 11-24.

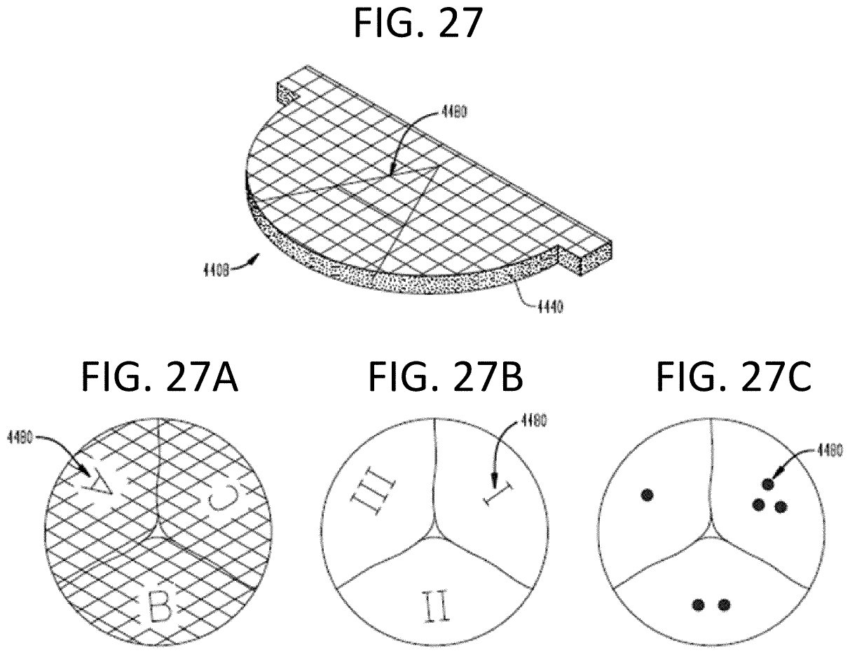

The leaflet 4308 in FIGS. 26 and 26A is similar in structure to the leaflet described in connection with FIG. 12, except that the polymer layer 4350 is disposed on the downstream surface 4315 of primary polymer layer 4340. In addition, leaflet 4308 contains one or more indicia 4380 that may be apparent visually to the naked eye or may be radiopaque to make it visible during surgery when the device is implanted within a patient's anatomy, or both. Indicia 4380 may help a surgeon position and orient the valve as needed and may assist in visualizing the movement of the leaflets to show an operable valve. Letters are used as the indicia 4380 in FIG. 26, but numbers, Roman numerals, symbols, or any other relevant indicia may be used as well. FIG. 26A is a view of a coapted set of leaflets, such as those shown in FIG. 3. It illustrates the use of a plurality of indicia 4380, one on each leaflet 4308. The indicia may be embedded within polymer layer 4350, may be sandwiched between adjacent polymer layers, or may be disposed between primary polymer layer 4340 and a coating forming layer 4350.

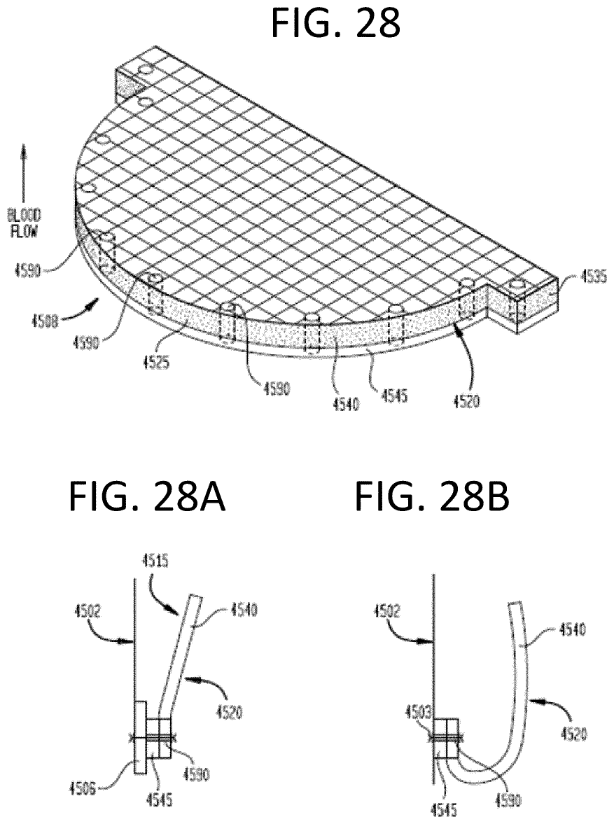

FIGS. 27, 27A, 27B, and 27C illustrate an embodiment similar to that shown in FIG. 26. Leaflet 4408, however, is composed entirely of an uncovered primary polymer layer 4440. The indicia 4480 may be melted or otherwise embedded into the polymer material, or glued or otherwise applied to a surface of the polymer layer and may be visual and/or radiopaque indicia. FIG. 27A shows an embodiment like that shown in FIG. 26A, in which indicia 4480 constitute a plurality of letters. FIG. 27B shows indicia 4480 as Roman numerals, and FIG. 27C shows the indicia as a series of dots.

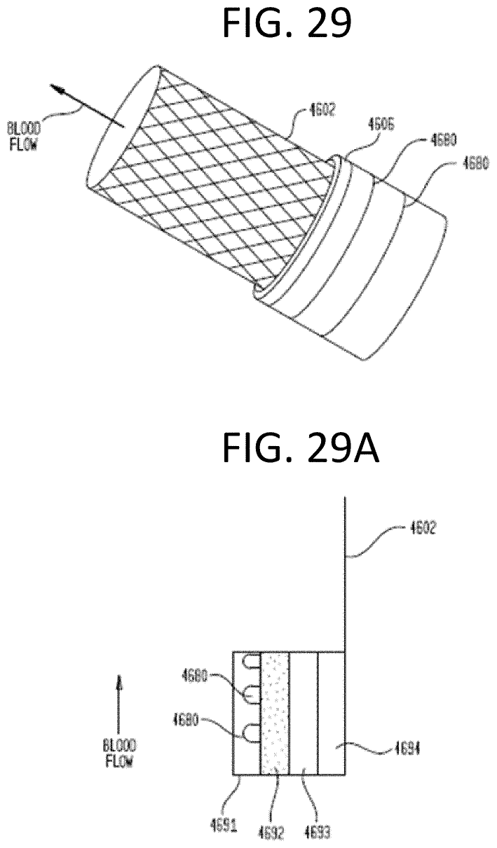

FIGS. 28, 28A and 28B illustrate a leaflet as previously described in connection with FIG. 18. Leaflet 4508 includes a primary polymer layer 4540 and attached to its upstream surface 4520 adjacent attachment edge 4525 is a partial layer 4545, which partially covers the upstream surface 4520 of the primary polymer layer. Additionally, leaflet 4508 includes a number of holes or grommets 4590 disposed adjacent attachment edge 4525 and through both primary polymer layer 4540 and partial layer 4545. These holes 4590 may facilitate suturing, lacing or other attachment of leaflet 4508 to the support structure. Holes 4590 may also be formed in the leaflet tabs 4535 to facilitate the attachment or lacing of the leaflet commissures to one another by aligning the holes in adjacent leaflet tabs, as well as the attachment of the leaflets to the stent. Holes 4590 may be formed by laser drilling, a process that locally melts the primary polymer layer and partial layer forming a smooth, tough, abrasion resistant surface, much like a grommet, that can provide resistance to damage caused by the passage of sutures therethrough during the suturing process. These holes or grommets 4590 may be coated with a more lubricous coating or polymer material that is permanent or one that can be removed to further improve the suturing process and prevent damage to the leaflet. Moreover, the laser drilling process may melt the various layers together in a localized area, which could help prevent fraying, delamination or damage. While laser drilled holes have been described, the holes may be produced by any other means as well, such as molding, mechanical or water jet drilling, and the like.

FIG. 28A shows a partial cross-sectional view of a stent with an attached valve assembly as previously described. In this view, stent 4502 has a cuff 4506 attached to its luminal surface. Leaflet 4508, composed of continuous polymer layer 4540, contains a partial layer 4545 on its downstream surface 4515, which is disposed between the cuff and the polymer layer 4540. Leaflet 4508 contains grommets 4590 through which the leaflet is sutured or laced to cuff 4506, stent 4502, or both. Grommets may also be formed in a pattern in cuff 4506 to facilitate the attachment of the cuff to the stent. FIG. 28B shows a similar arrangement in which the device contains no cuff and a partial layer 4545 is disposed on the upstream side 4520 of leaflet 4508, as in FIG. 28. Grommets 4590 are provided through both the continuous polymer layer 4540 and the partial layer 4545, enabling the leaflet to be sutured or laced via suture 4503 to the stent 4502. Grommets have been described here in connection with leaflets having a partial layer. However, these grommets could be formed in fully covered or completely uncovered leaflets, in covered or uncovered cuffs, and in any portion of a medical device that may be attached to a support structure by a suture.

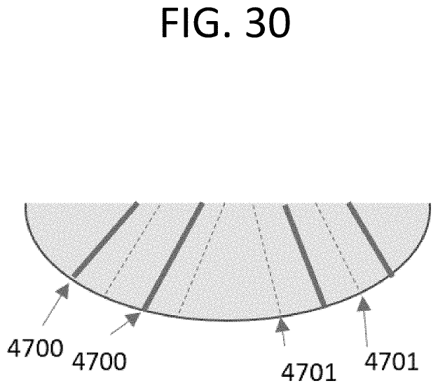

FIG. 29 illustrates a stent 4602 containing a cuff 4606 on its abluminal or exterior surface. Cuff 4606 contains a plurality of indicia, in this case, radiopaque bands 4680 disposed at various intervals to assist the surgeon in placement of the prosthetic valve. The structure of cuff 4606 is illustrated in more detail in FIG. 29A. Attached to the exterior of stent 4602, and provided as an illustrative example only, cuff 4606 has four layers. The outermost layer 4691 is a polymer layer covering the entire exterior surface of cuff 4606. The next innermost layer is a polymer layer 4692. Disposed between the polymer layer 4692 and outermost polymer layer 4691 are circumferential radiopaque and/or visual indicia 4680. Between the polymer layer 4692 and stent 4602 are two additional polymer layers 4693 and 4694. Each of layers 4691, 4692, 4693 and 4694 may be composed of the same or different polymer materials and/or may have the same or different dimensions and thicknesses as previously described in connection with the leaflets of FIGS. 11-28.

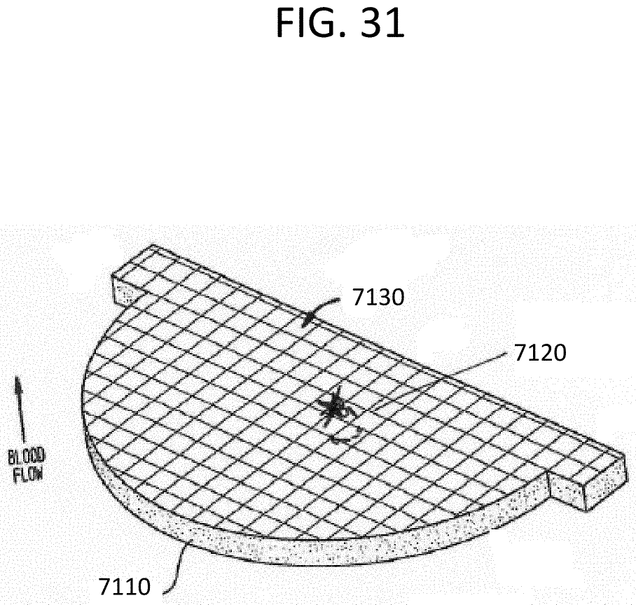

In an additional embodiment, the method for forming a leaflet for use in a prosthetic heart valve includes folding a first portion of a sheet of material over a second portion of the sheet of material and repeating the folding step until the leaflet has a selected thickness. Another embodiment of the present disclosure provides a leaflet for use in a prosthetic heart valve including a pleat 4700 in a sewing region of the leaflet, as shown in FIG. 30. The pleat unfolds when a load is exerted on the leaflet during loading and reforms during valve opening. The pleat may be formed by folding a first portion of a polyurethane sheet over a second portion of the polyurethane sheet. The ultimate goal of using one or more pleats is to reduce strain in the sheet material. It is based on the way that natural collagen fibers uncrimp during initial loading. This is typically strain at low stresses; after the fibers are uncrimped, the fibers reach higher stresses quicker. Pleats can be folded into the structure such that they fully expand to their original structure once deployed. A folded pleat 4701 is also illustrated in FIG. 30. One end of the pleat may also be tacked down by spot welding or using a suture or grommet such that while expanded, the pleat maintains its shape to some degree. Pleats may also be facilitated by scoring or partial ablation to promote folding.

Using polyurethane or other polymer films as an example, the polymer sheet materials may comprise a single polymer layer. It may be folded back on itself once or multiple times to achieve a desired thickness, thus forming a multilayered sheet. This is an additional method of producing a multi-layered sheet material in accordance with the invention. And, a single layer may be folded around and intermediate film of the same or different composition. In the alternative, a sheet so folded could have applied to one or more of its major surfaces, additional layers by applying a film or coating.

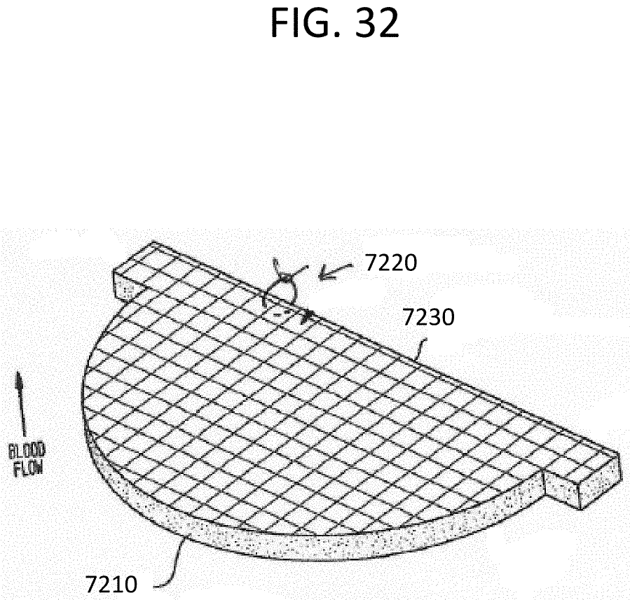

FIG. 31 shows a leaflet 7110 formed from a polymer sheet material according to the present disclosure including a single stitch 7120 disposed through a major surface 7130 of the leaflet.

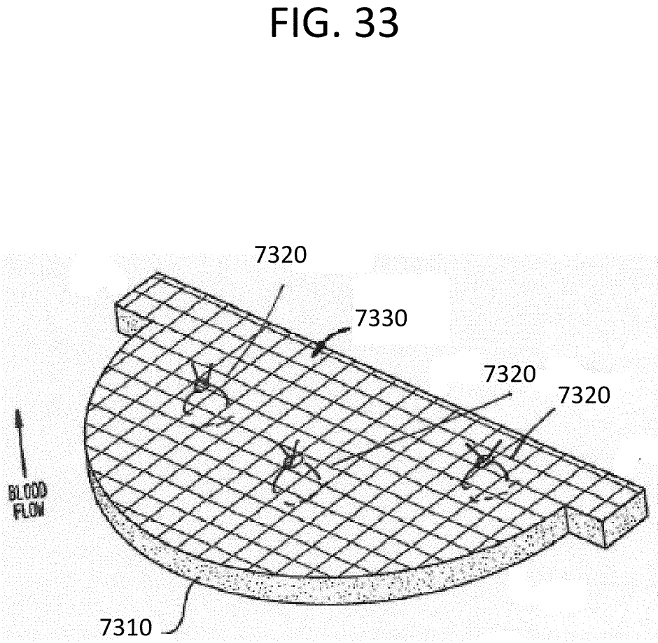

FIG. 32 shows a leaflet 7210 of a leaflet formed from a polymer sheet material according to the present disclosure including a single stitch 7220 in the free edge 7230 of the leaflet.

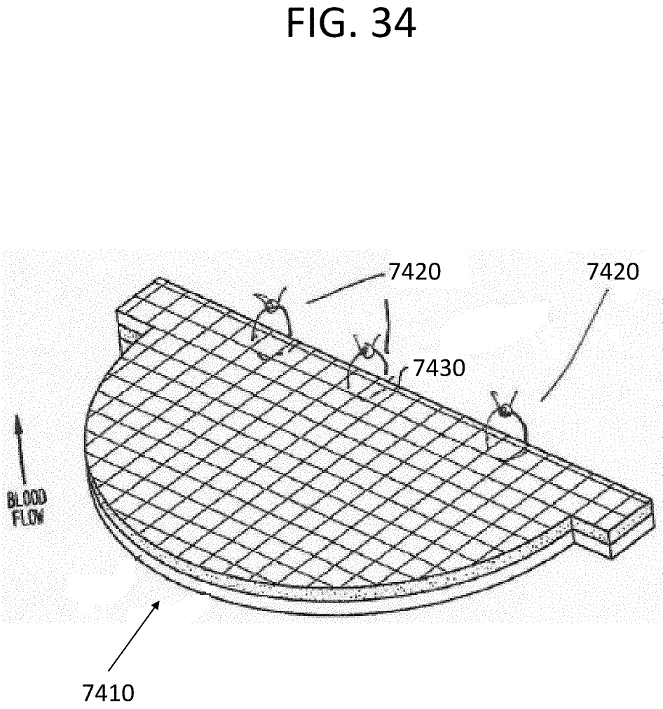

FIG. 33 shows a leaflet 7310 of a leaflet formed from a polymer sheet material according to the present disclosure including multiple stitches 7320 on a major surface 7330 of the leaflet.

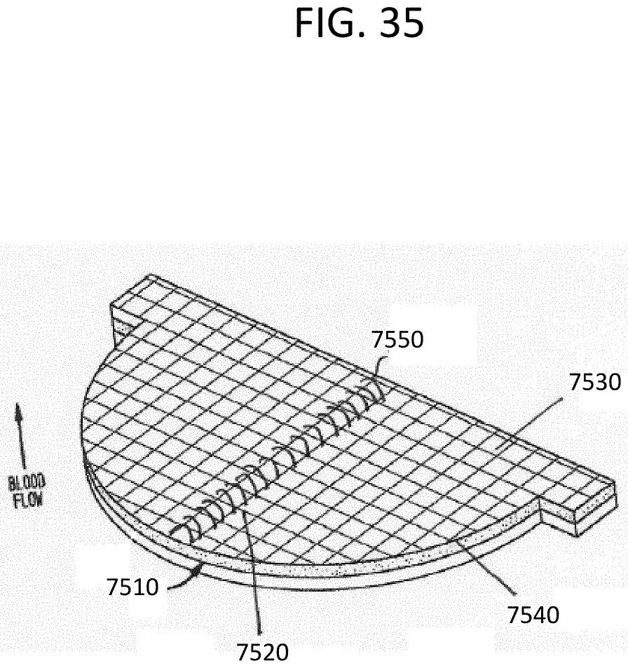

FIG. 34 shows a leaflet 7410 formed from a polymer sheet material according to the present disclosure including multiple stitches 7420 along the free edge 7430 of the leaflet. Leaflet 7410 is illustrated with a plurality of layers. Stitches 7420 may pass through one layer or all of the layers.

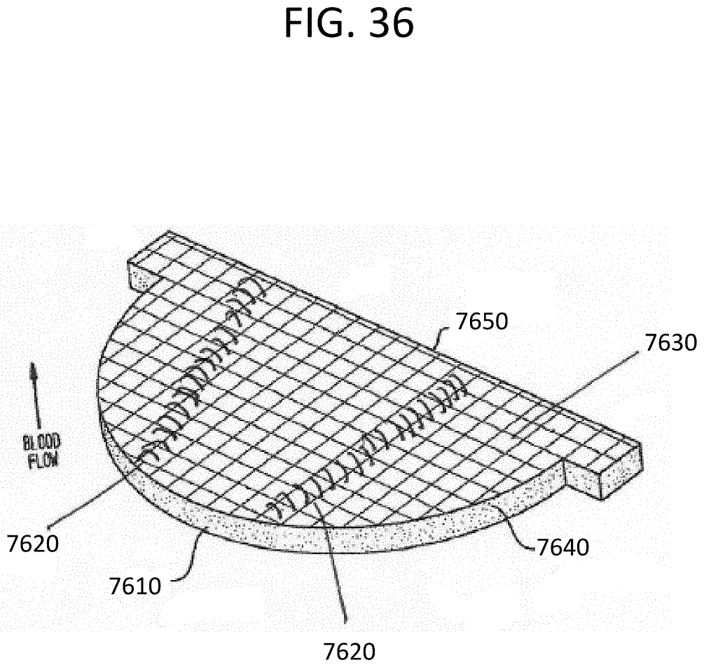

FIG. 35 shows a leaflet 7510 formed from a polymer sheet material according to the present disclosure including a suture line 7520 extending across a major surface 7530 of the leaflet from the attachment edge 7540 to the free edge 7550. Leaflet 7510 is illustrated with multiple layers as noted in FIG 34. Suture line 7520 may pass through the one or all of the layers. By varying the density and number of the stitches, by varying their relative width, and by using sutures of different materials and constructions, one can impart varying degrees of reinforcement, impart or preserve a three dimensional shape, such as the “belly” or “spinnaker” like shape of a native leaflet, or may bias the leaflet into a closed position.

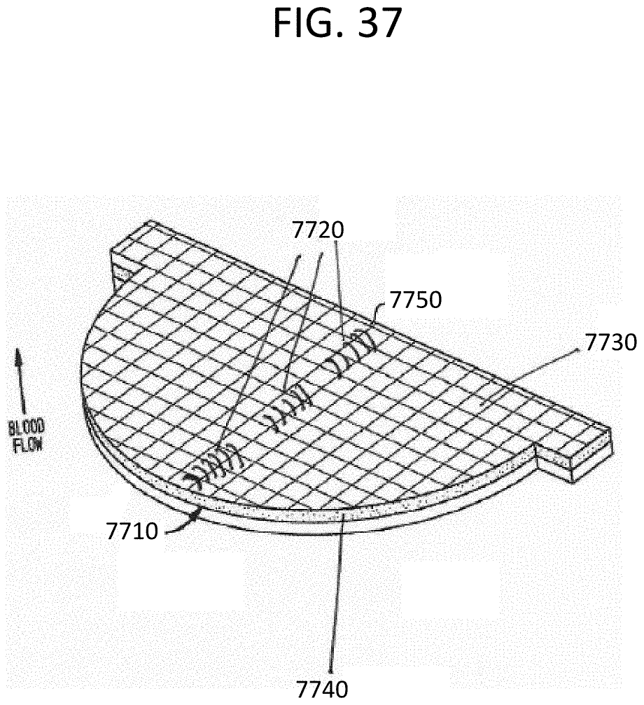

FIG. 36 shows a leaflet 7610 formed from a polymer sheet material according to the present disclosure including a plurality of suture lines 7620 extending across a major surface 7630 of the leaflet from the attachment edge 7640 to the free edge 7650.

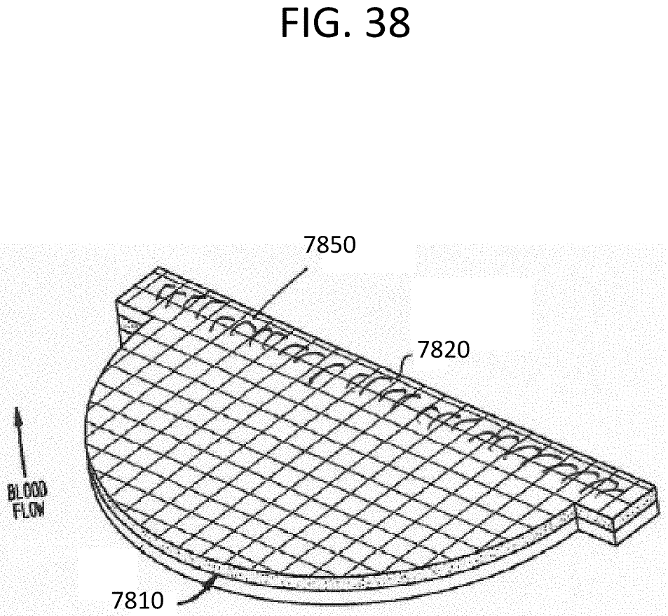

FIG. 37 shows a leaflet 7710 formed from a polymer sheet material according to the present disclosure including a discontinuous suture line 7720 extending across a major surface 7730 of the leaflet from the attachment edge 7740 to the free edge 7750. Leaflet 7710 is illustrated with a plurality of layers. Suture line 7720 may pass through one or all of the layers. In addition to the functions described in connection with FIG. 35, the use of discontinuous sutures can create regions of relatively greater and lesser flexibility along the suture line.

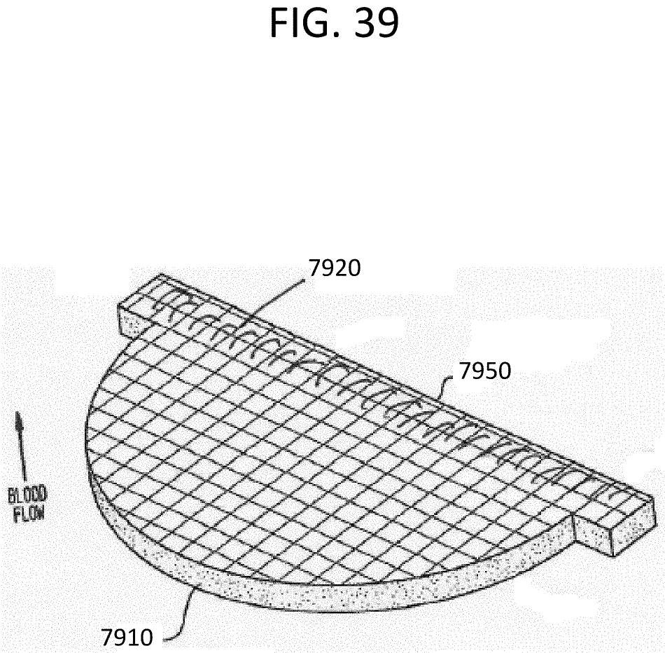

FIG. 38 shows a leaflet 7810 formed from a polymer sheet material according to the present disclosure including a suture line 7820 extending along the free edge 7850 of the leaflet. Leaflet 7810 is illustrated with a plurality of layers. Suture line 7820 may pass through one or all of the layers.

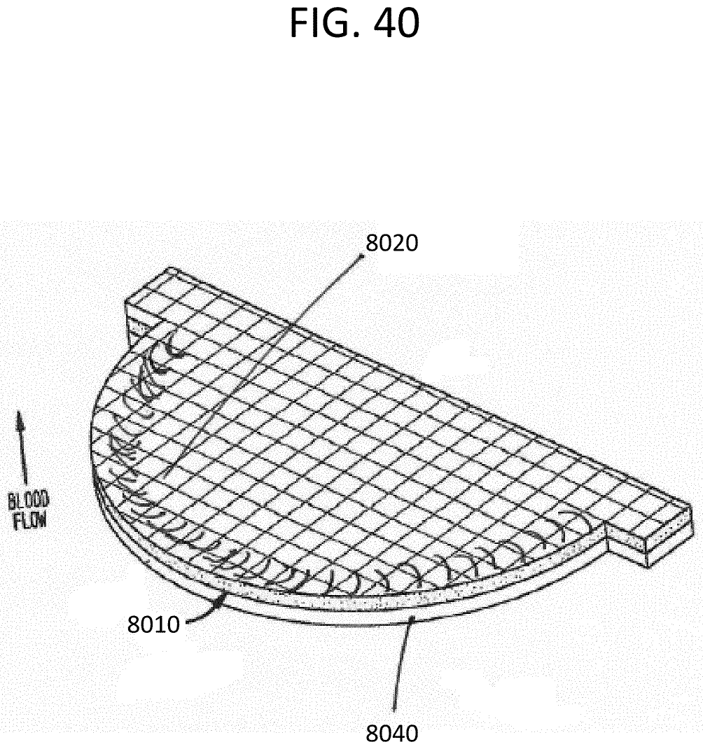

FIG. 39 shows a leaflet 7910 formed from a polymer sheet material according to the present disclosure including a discontinuous suture line 7920 extending along the free edge 7950 of the leaflet.

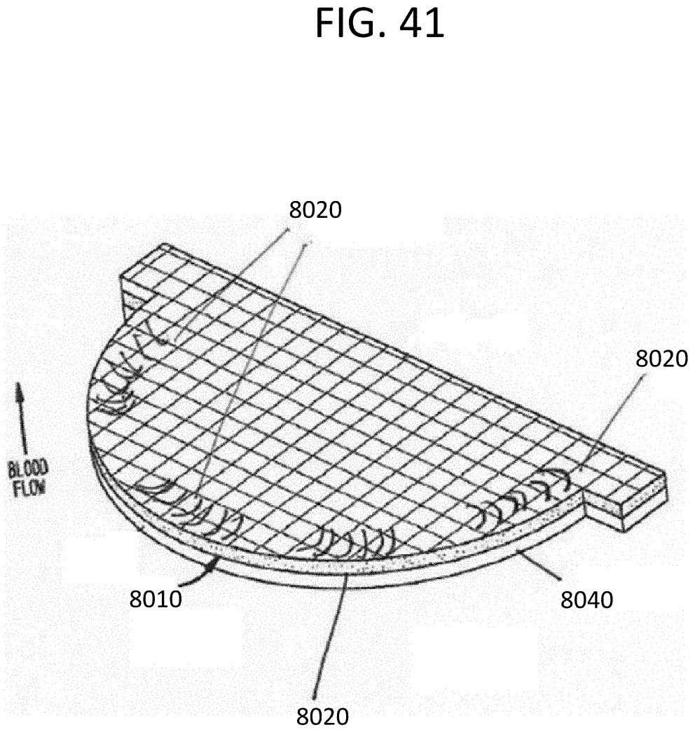

FIG. 40 shows a leaflet 8010 formed from a polymer sheet material according to the present disclosure including a suture line 8020 extending along the attachment edge 8040 of the leaflet. In addition to the functions noted above for suture lines, this suture line can in addition or instead provide benefits in attaching the leaflet by providing reinforcement through which attachment sutures will pass. It could also assist in protecting the edge. The suture line could extend through one or more layers provided.

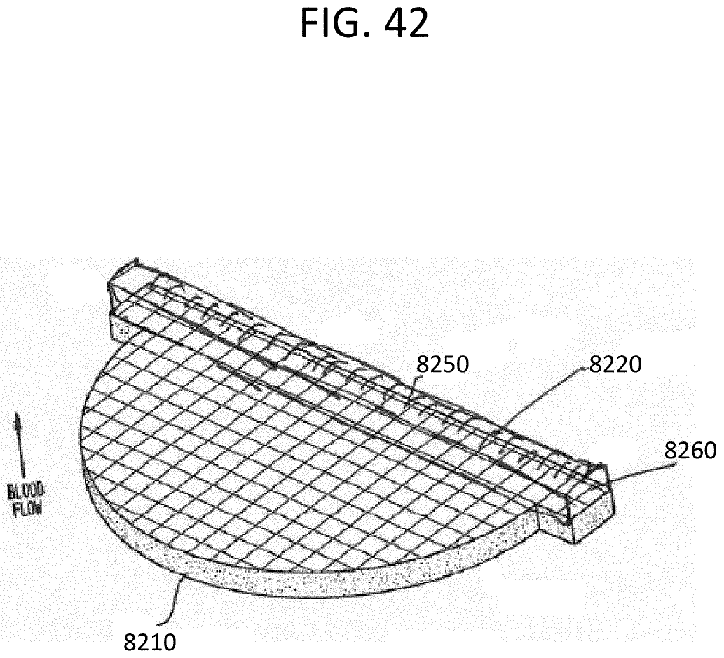

FIG. 41 shows a leaflet 8110 formed from a polymer sheet material according to the present disclosure including a discontinuous suture line 8020 extending along the attachment edge 8040 of the leaflet. Leaflet 8010 is illustrated with a plurality of layers and suture lines 8020 may pass through one or more of those layers. As also described in connection with other suture lines, the gaps here can provide regions of relative flexibility.

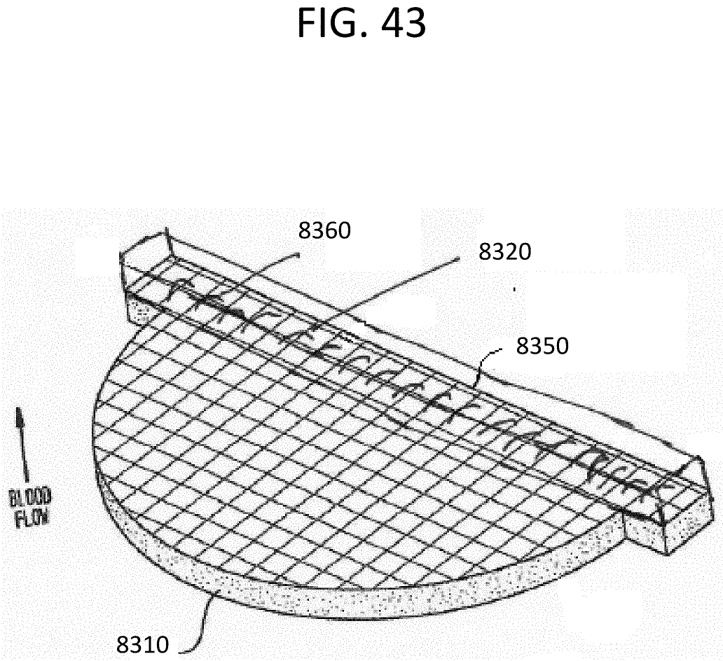

FIG. 42 shows a leaflet 8210 formed from a polymer sheet material according to the present disclosure including a suture line 8220 extending along the free edge 8250 of the leaflet through a partial coating layer 8260 as well as the layer it covers.

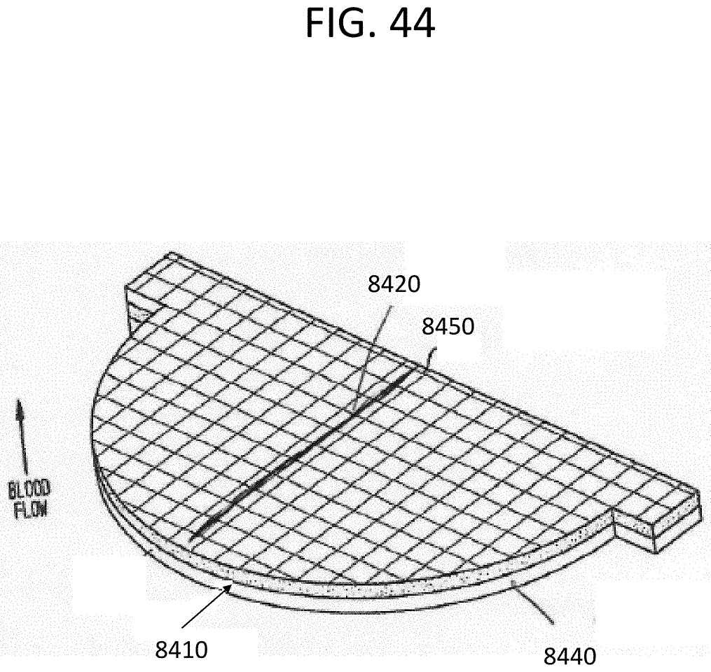

FIG. 43 shows a leaflet 8310 formed from a polymer sheet material according to the present disclosure including a suture line 8320 extending along the free edge 8350 of a leaflet, the suture line being disposed underneath a partial coating layer 8360 and laced through the polymer layer.

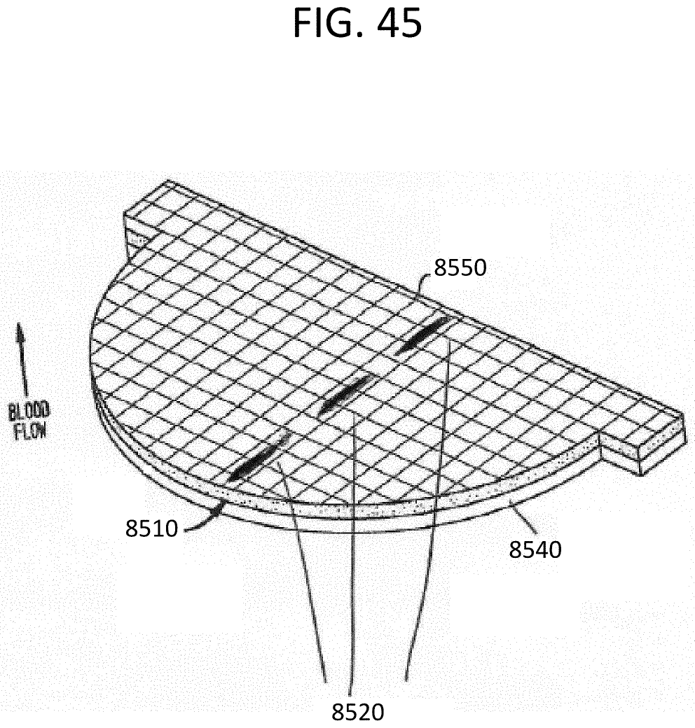

FIG. 44 shows a leaflet 8410 formed from a polymer sheet material according to the present disclosure including a wire 8420 extending from the attachment edge 8440 to the free edge 8450 of the leaflet. Leaflet 8410 is illustrated with a plurality of layers. Wire 8420 may be positioned through a major surface of a layer, disposed within a layer, or positioned between layers.

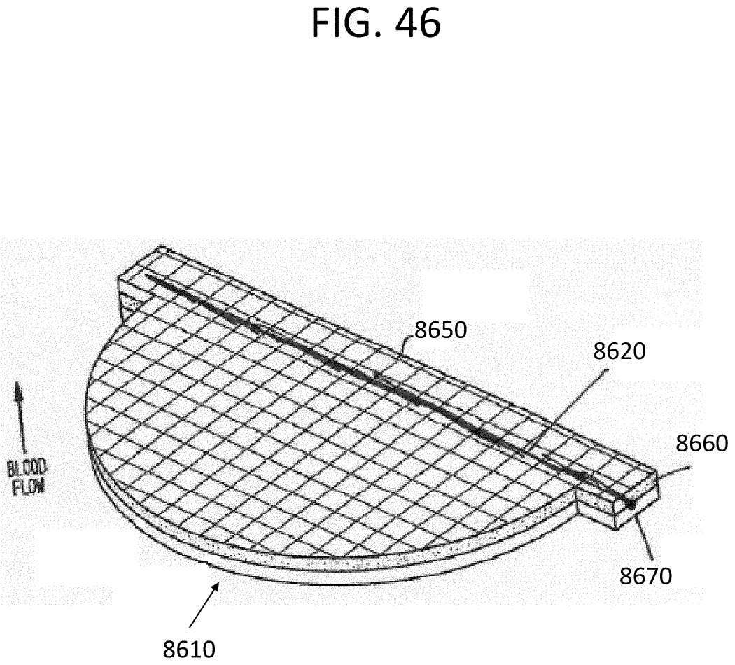

FIG. 45 shows a leaflet 8510 formed from a polymer sheet material according to the present disclosure including a discontinuous wire 8520 extending from the attachment edge 8540 to the free edge 8550 of the leaflet. Leaflet 8510 is illustrated with a plurality of layers. Wire 8520 may be positioned on a major surface of a layer, disposed within a layer, or between layers.

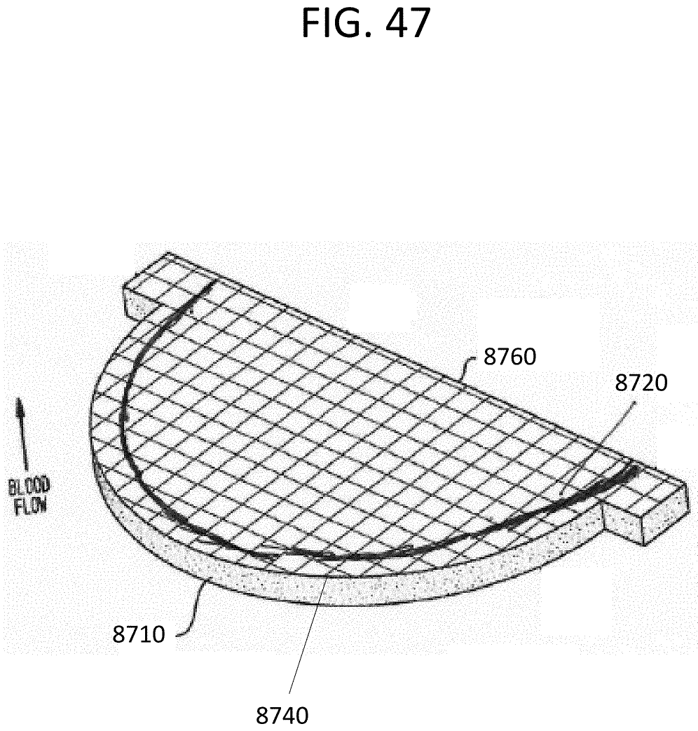

FIG. 46 shows a leaflet 8610 formed from a polymer sheet material according to the present disclosure including a wire 8620 extending across a major surface of the leaflet and along the free edge 8650 of the leaflet, in this case, the wire being disposed between the illustrated polymer layers layer 8660 and 8670. In other embodiments, the wire 8620 could be disposed on a major surface of one the layers or disposed within a layer.

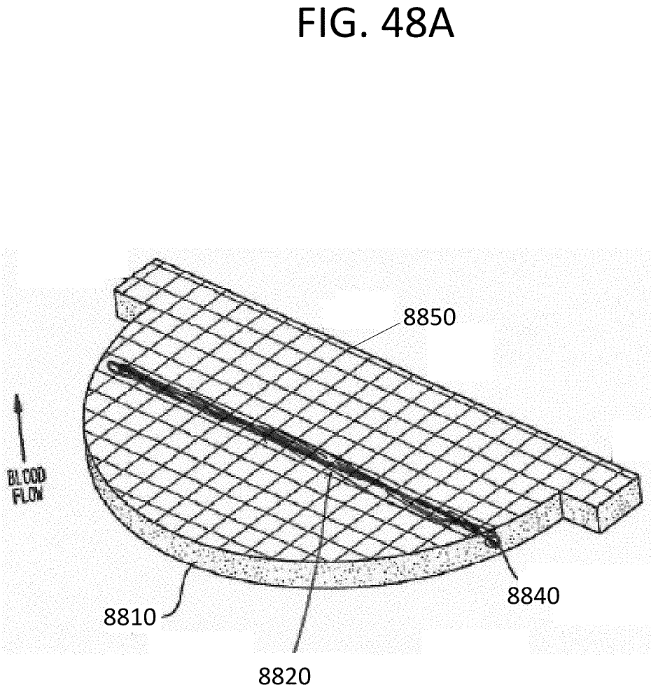

FIG. 47 shows a leaflet 8710 formed from a polymer sheet material according to the present disclosure including a wire 8720 extending across a major surface of the leaflet and along the attachment edge 8740 of the leaflet. In the illustrated embodiment, the ends of the wire 8720 extend to the free edge 8760, although in other embodiments the ends of the wire may be spaced a distance from the free edge.

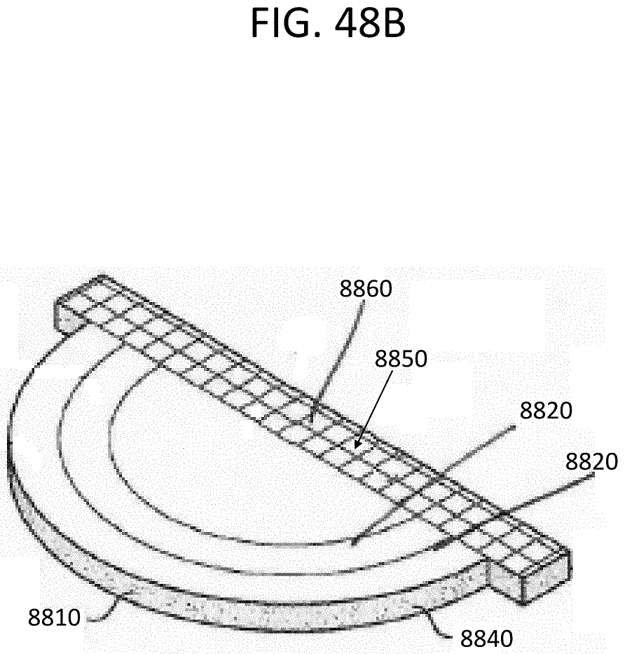

FIG. 48A illustrates the use of a wire 8820 adhered to a major surface of a polymer layer spaced apart from, but aligned roughly parallel to the free edge 8850 of leaflet 8810. Both of the free ends of this wire intersect the attachment edge 8840. One such wire is shown but other rows of wire, continuous or discontinuous, may also be used. Instead of a wire, as illustrated, this could also be accomplished with a series of stitches or suture line(s). As shown in FIG. 48B, several of these structures 8820 could also be placed roughly conforming to or paralleling the contour of the attachment edge 8840 spaced apart from each other. In this illustration, FIG. 48B, two such wires 8820 are disposed in a roughly concentric pattern with their ends running between the continuous or complete polymer layer and a partial layer 8860 disposed at the free edge 8850 of leaflet 8810.

Various medical devices, including collapsible and expandable prosthetic heart valves, have been described above which may incorporate the polymer sheets described herein. Although the prosthetic collapsible and expandable heart valves have generally been described in connection with a prosthetic aortic valve, those heart valves may be designed for replacing any heart valve. For example, collapsible and expandable prosthetic mitral valves may include an outer stent portion to anchor into the mitral valve annulus, and an inner stent portion to house the prosthetic leaflets, with the inner stent connected to the outer stent, for example so that the inner stent is substantially mechanically isolated from the outer stent. In such embodiments, the outer stent portion may include features to help secure the prosthetic mitral valve within the mitral valve annulus, and the inner stent portion may be substantially cylindrical (e.g. a right cylinder) so that the valve assembly may have a generally circular profile in cross section. The sheet materials described herein may be used for prosthetic leaflets of the prosthetic mitral valve and/or for any cuffs or skirts on the prosthetic mitral valve, which may include inner cuffs and/or outer cuffs of the inner stent portion and the outer stent portion, as well as any other cuff or skirt portions, such as cuff portions that connect the inner stent portion to the outer stent portion. Prosthetic mitral valves having inner and outer stent portions are described in greater detail, for example, in U.S. Patent Publication Nos. 2017/0196688 and 2019/0328525, and U.S. Pat. No. 10,052,204, the disclosures of which are hereby incorporated by reference herein.

The use of polymer sheet materials having complete or continuous layers, complete or continuous patterned layers or partial layers to provide abrasion resistance to the free edge of leaflets, to help facilitate the attachment of the leaflets to a supporting structure by reinforcing and preventing the unraveling or delamination of attachment edges, to provide reinforcing structures, folding zones, etc., and to provide indicia, have been described mainly in terms of leaflets and, to a lesser extent, cuffs designed for use in collapsible/expandable prosthetic heart valves. However, these described structures may be incorporated in polymer sheet materials for use in other types of collapsible/expandable valves. They may be used as well in constructing leaflets and cuffs or other structures for surgical heart valves—those sewn in place using open heart surgery. And they may be used in other medical devices as described herein.

One sheet material which may be useful for some applications is composed of five layers, two polymer layers (each about 20 μm thick) laminated to one side of a primary polymer layer and two other polymer layers (each about 20 μm thick) laminated to the other side of the same primary layer. The outer two polymer layers may, for example, be made of Dyneema Purity® membrane 55501 available from DSM Biomedical (www.dsmbiomedical.com). Dyneema Purity® membrane 55501 is composed of UHMWPE and is said to be known for uses in the medical device industry. The properties of Dyneema Purity® membrane 55501 are specified in its Product Data Sheet from DSM Biomedical dated June 2015. Other materials, a greater or lesser number of layers, layers of variable thicknesses, and the like may be used instead. This same material could be used for the primary polymer layer, or another polymer layer, either of UHMWPE or some other material, or something that is stretched or directionally oriented, may be used for the primary layer.

After the desired polymer sheet material has been created and elements shaped or cut from the sheets (assuming that the entire sheet is not used or that the component is created from pre-shaped components—all of which are contemplated), it will typically need to be connected to a supporting structure (such as a stent if the material is intended for use as a cuff and/or leaflets of a prosthetic heart valve). The attachment may be accomplished through any one of a number of suitable methods, including suturing, heat bonding, gluing, wrapping, electrospinning, laminating, mechanical attachment such as hooks, hook-and-loop fasteners, sandwiching between two supporting structures, or bonding directly to the supporting structure, such as integrating the polymer sheet materials to the supporting structure while the supporting structure is in a non-set state (e.g., a liquid) in which curing of the supporting structure results.

One aspect of the disclosure is a collapsible/expandable heart valve which may be implanted through a catheter or trocar, the heart valve including a valve assembly comprising a polymer sheet material as described herein, and in particular, a heart valve in which the sheet material is used to form the leaflets and/or cuffs shown in FIGS. 11-29A. In one such embodiment, the outer cuff may be made of a polymer sheet material of the disclosure. In another such embodiment, the inner cuff may be made of a polymer sheet material of the disclosure. In still another such embodiment, both the inner and outer cuffs may be made of a polymer sheet material of the disclosure.

In another embodiment, at least one leaflet may be made from a polymer sheet material in accordance with the disclosure. In another embodiment, some, but not all of the leaflets may be made from a polymer sheet material in accordance with the disclosure. It is also contemplated that all leaflets may be produced from a polymer sheet material in accordance with the disclosure. In one desirable embodiment, all of the leaflets may be made of the same polymer sheet material in accordance with the disclosure.

It is also an embodiment of this aspect of the disclosure that at least one cuff and at least one leaflet of the valve assembly may be composed of a polymer sheet material in accordance with the disclosure In one further embodiment, both the at least one cuff and the at least one leaflet of the valve assembly may be made of the same polymer sheet material in accordance with the disclosure. In another embodiment, all of the cuffs and all of the leaflets may be made from polymer sheet materials in accordance with the disclosure.

While the disclosure above provides for the use of polymer sheet materials in accordance with the disclosure for prosthetic leaflets, inner cuffs, and/or outer cuffs of collapsible/expandable and surgical prosthetic cardiac valves, the concepts may be similarly or identically applied to other prosthetic valves, such as prosthetic venous valves. Prosthetic venous valves may have generally similar structures and components as those described for the prosthetic heart valves, including a stent, one or more prosthetic leaflets, and optionally inner and/or outer cuffs. If the stent is self-expandable or balloon expandable, the stent may maintain a desired position within the vasculature via a friction fit. If the stent is non-collapsible, it may be sutured or otherwise fixed at the desired position within the vasculature. The one or more prosthetic leaflets may be coupled to the stent and/or to an inner and/or outer cuff attached to the stent, for example via sutures. The prosthetic leaflets may allow blood to flow in substantially only one direction within the vasculature. The inner and/or outer cuffs may assist in enhancing sealing to help prevent blood from flowing in the retrograde direction past the valve, and may also aid in coupling the one or more prosthetic leaflets to the stent. The prosthetic leaflets, inner cuffs, and outer cuffs of the prosthetic venous valves may be formed of any of the polymer sheet materials described above for components similar to those of the prosthetic cardiac valves.

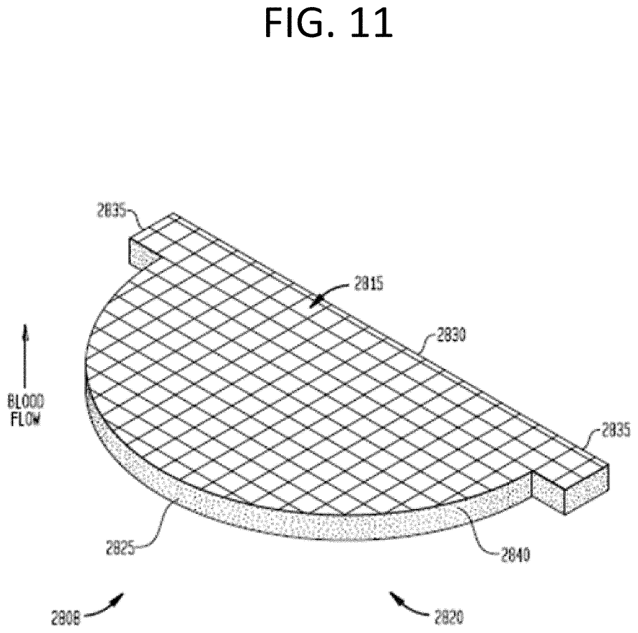

The polymer sheet materials in accordance with the disclosure herein may have still further applications, for example with occluders, which may also be referred to as closure devices. Such occluders may be used to treat any suitable abnormality or condition, including patent foramen ovale (“PFO”), atrial septal defect (“ASD”), ventricular septal defect (“VSD”), patent ductus arteriosus (“PDA”), and left atrial appendage (“LAA”) closure. Occluders may have various different configurations depending on factors such as the type of abnormality to be occluded, the location of the target site, the condition of the patient's vasculature or cardiac anatomy, and the practitioner's preferences. The occluders described herein have a collapsed condition and an expanded condition. For example, in the embodiment shown in FIG. 10A, a closure device 2000 has a first expanded volume portion 2010 and a second expanded volume portion 2020 that are substantially perpendicular to a central axis extending along closure device 2000. The first expanded volume portion 2010 may be proximate a first end of closure device 2000, with the second expanded volume portion 2020 spaced axially from the first expanded volume portion 2010 and proximate a second end of closure device 2000. The first expanded volume portion 2010 may be connected to the second expanded volume portion 2020 via an axial portion 2030.

As depicted in FIG. 10A, the first expanded volume portion 2010 in the expanded condition may have the shape of a thin disk, and is intended to help maintain the closure device 2000 in position at the target site, as described in greater detail below. The second expanded volume portion 2020 in the expanded condition may, in some cases, be a generally cylindrical body that is substantially thicker in the axial direction than the first expanded volume portion 2010 and axially disposed toward the second end. The second expanded volume portion 2020 when expanded may be sized to be somewhat larger in diameter (e.g., about 10-30% larger) than the inside diameter of the vessel, cavity, or lumen to be occluded to facilitate anchoring of the device to prevent dislodgement, but not so large when collapsed as to not fit in the vessel, cavity or lumen.

At the same time, in the expanded condition, the first expanded volume portion 2010 of the closure device 2000 may have a diameter that is larger than the diameter of the second expanded volume portion 2020. This larger diameter is intended to abut the wall surrounding the abnormal aperture to prevent device movement further into the aperture and to assist in sealing the aperture. For example, the first expanded volume portion 2010 may be oversized so as to overlie the ostium or opening of the LAA in a position adjacent to, and in flush contact with, the wall of the atrium. The first expanded volume portion 2010 may also be flexible so as to be capable of conforming to the curvature of the wall of the atrium in LAA applications or other cardiac or vascular structures in other applications. Although one configuration of the first and second expanded volume portions 2010, 2020 is described above and shown in the figures, various other configurations and sizes may be used depending on the particular application or condition to be treated. For example, one or both expanded volume portions 2010, 2020 may be thin disks or disks having a convex distal end, or the device may include a smaller diameter cylindrical portion between two larger diameter disks. Moreover, the depth or thickness of the first and/or second expanded volume portions may depend on the thickness and number of layers used to make the closure device 2000.

The first expanded volume portion 2010, the second expanded volume portion 2020, and the axial portion 2030 may each be formed of a shape-memory alloy, such as braided nitinol, to facilitate collapsing the closure device 2000 for minimally invasive delivery, and to facilitate expansion to a pre-set shape upon delivery of the closure device 2000 to the intended location. A first coupling 2015 may be disposed adjacent the first expanded volume portion 2010 and may enable connection of a delivery device or other device to closure device 2000. For example, first coupling 2015 may include internal or external threads that mate with corresponding threads of another device. A second coupling 2025, similar to the first coupling 2015, may be disposed adjacent to or within the second expanded volume portion 2020. Second coupling 2025 may also include internal or external threads for connection to corresponding threads of another device. It should be understood that other coupling mechanisms, such as press-fit or snap-fit arrangements, may be utilized in first and second couplings 2015, 2025. Additional details of closure device 2000 and similar devices are described in U.S. Pat. No. 8,758,389, the disclosure of which is hereby incorporated by reference herein.

FIG. 10B is a schematic view of closure device 2000 positioned within the LAA of a left atrium LA. In patients with certain conditions, such as atrial fibrillation, blood clots may tend to form in the LAA. Implanting a device such as closure device 2000 may lead to partial or complete occlusion of the LAA, thus reducing the risk of thrombi breaking off the LAA and entering the blood stream. In order to help better occlude the LAA, it may be desirable to include a polymer sheet component on the interior surface, exterior surface, or both surfaces of the closure device 2000. For example, part or all of the outer surface, and/or part or all of the inner surface, of closure device 2000 may include one or more layers of the polymer sheet materials described herein. Such sheets may help to better and/or more quickly occlude the LAA. In some embodiments, if portions of closure device 2000 are formed of two or more layers of braided metal, such as braided nitinol, polymer sheet materials in accordance with the disclosure may be included between the two or more layers of braided metal. Other closure devices, such as PFO closure devices, may similarly include polymer sheet material in accordance with the disclosure on part or all of an exterior surface and/or on part or all of an interior surface (and/or between multiple layers of braided mesh if present), for similar purposes as described in connection with closure device 2000.

The polymer sheet materials in accordance with the present disclosure may also be used to form the entirety, or portions, of various types of prosthetic vascular conduits. For example, a prosthetic aortic graft may be implanted into the aorta to treat a weakened portion of the aorta resulting from a thoracic aneurysm. Prosthetic vascular conduits may be used to perform a bypass to reroute the path of blood flow, for example as a lower extremity bypass, a cardiac bypass in conjunction with open heart surgery, or to serve as an access point to the circulatory system, such as for hemodialysis. Prosthetic vascular conduits may also be used as arteriovenous (“AV”) shunts. AV fistulas are abnormal connections between an artery and vein, although they may be surgically created in order to assist with hemodialysis treatment. When an AV fistula is surgically created, an AV shunt formed from a polymer sheet material in accordance with the present disclosure may be implanted to provide the desired connection between the artery and vein. Prosthetic vascular conduits are typically cylindrical in shape and have been formed of PTFE or Dacron. However, prosthetic vascular grafts may instead be formed of a polymer sheet material as described herein.

In addition to the above uses, the polymer sheet materials described herein may have additional uses. For example, hernias occur when there is an opening or a weakness in the muscle and/or connective tissue through which organs begin to push. Hernias are frequently treated with a fabric mesh that provides closure and support of the weakness and/or opening that forms the hernia. The mesh acts to patch the hernia, and is frequently formed of a plastic material. Such patches may instead be formed of a polymer sheet material in accordance with the disclosure herein, whether the patches are continuous or formed as a mesh. Indeed, a mesh can be formed from the sheet material by cutting one or more holes or openings through the entire thickness of the sheet. By cutting a number of such holes or openings, a mesh can be created. A mesh could also be created by making a number of perforations and then stretching the material such that the opposed edges of the perforations are drawn apart, forming openings. And while hernia repair is one exemplary use of patches formed of a polymer sheet material in accordance with the disclosure herein, such patches may be used in any other suitable procedure, including skin patches, vaginal patches, and/or cardiac patches to provide the desired support to the underlying anatomy.

In some embodiments, the polymer sheet materials described herein may be used to form adhesion barriers. Adhesion barriers are medical implants that may be used to reduce abnormal internal scarring following surgery. The polymer sheet materials forming the adhesion barriers may act to separate internal tissues and/or organs while they heal post-surgery.

While the above-described embodiments of devices that incorporate the polymer sheet materials described herein are generally directed to devices intended to be permanently implanted into the body, the polymer sheet materials may be used for various types of medical devices that are used in medical procedures, but not intended to be implanted at all, or not intended to be implanted for longer than the surgical procedure. One such example is an embolic protection device. Generally, an embolic protection device may be used to prevent emboli that are dislodged during a medical procedure from entering the vasculature. Typically, embolic protection devices either capture dislodged emboli so that the emboli can be removed from the body, or otherwise deflect emboli from entering high-risk vasculature (such as the carotid arteries) so that the emboli are able to pass through the vasculature where there may be a lower risk of complications from the emboli. Embolic protection devices may include various types of filters that allow blood to pass through the filter, but are formed as meshes or with pore sizes small enough to trap emboli therein, or otherwise to deflect emboli. Such embolic protection devices may be formed of the polymer sheet materials described herein. Examples of embolic protection devices are disclosed in greater detail in U.S. Patent Pub. Nos. 2014/0249567 and 2018/0116780, the disclosures of which are hereby incorporated by reference herein.

While the polymer sheet materials described herein may be used with short-term filters such as those described immediately above, they may also be used in permanently implanted filters, such as inferior vena cava (“IVC”) filters, whether or not the IVC filter is intended to be retrievable. IVC filters typically have a central base and a plurality of legs that extend outwardly from the base to form an overall conical shape, with the legs intended to make contact with the interior surface of the lumen of the IVC to help support the IVC filter in place. The IVC filter functions by allowing blood to flow around the filter, while trapping emboli that pass into the filter, preventing the emboli from causing blockages in the vasculature downstream of the IVC filter. The IVC filters may be formed of a metal or other biocompatible material and the polymer sheet materials described herein may encapsulate portions or all of the IVC filter, or in other embodiments the IVC filter may be formed entirely of the polymer sheet materials described herein. It should be understood that for IVC filters, or any other application disclosed herein, specific parameters of the disclosed polymer sheet materials, such as dimensions, as well as fabrication methods, may be altered to suit the particular application.

The polymer sheet materials useful in any of the medical devices described or contemplated may be made by any known method used to form polymer sheets. These include, without limitation, mechanical methods, for example cutting with scissors or a blade. Other techniques include, for example, cautery; mechanical cutting, stamping; chemical, laser, ultrasonic, or water jet cutting, bio-glue, gluing, reacting, crosslinking, folding, sewing, riveting, spot welding or laminating.

These fabrication methods include cutting a component such as a leaflet shaped structure from a single layer or multiple layer polymer sheet. Alternatively, the method includes forming a sheet material in the desired shape by molding a liquid or molten polymer material and allowing it to form a sheet material in the desired shape. Sheets of polymers can be produced by melting fiber mats so they form a continuous layer, by layering tapes, by extrusion methods and the like. See, for example, U.S. Pat. Nos. 4,876,049; 7,923,094; US Patent Publication No. 2008/0020182 A1; and EP 2,435,250 A1.

According to an aspect of the disclosure, a prosthetic heart valve comprises: an expandable stent extending in a longitudinal direction between an inflow end and an outflow end; a cuff coupled to a luminal surface of the stent; and a plurality of prosthetic leaflets coupled to at least one of the cuff and the stent and having an open condition and a closed condition, the plurality of prosthetic leaflets adapted to allow blood to flow from the inflow end toward the outflow end when in the open condition and to retard blood from flowing from the outflow end toward the inflow end when in the closed condition, each of the plurality of leaflets being formed of a polymer sheet material in accordance with this disclosure including at least one layer of ultra-high molecular weight polyethylene (UHMWPE) or polyethylene such as PTFE, having a thickness of between about 50 μm and about 100 μm; and in some embodiments, a single layer thickness of 10-30 and in still other embodiments, 15-20 μm.

According to another aspect of the disclosure, a prosthetic heart valve comprises: an expandable stent having a luminal surface; and a valve assembly attached to the luminal surface of the stent, the valve assembly including a cuff and a leaflet, the leaflet having a first major surface, an opposed second major surface, an attachment edge, a free edge, and a plurality of tabs, both the cuff and the leaflet composed of a polymer sheet material composed of at least one layer and at least one of the layers, partial layers, coatings or partial coatings is composed of one or more of the polymers selected from the group consisting of polytetrafluoroethylene (“PTFE”); low density PTFE; high density PTFE; ultra-high molecular weight PTFE (“UHMWPTFE”); stretched PTFE; expanded PTFE; polyethylene (“PE”); low density PE; high density PE; ultra-high molecular weight PE (“UHMWPE”); polypropylene (“PP”); low density PP; high density PP, ultra-high molecular weight PP (“UHMWPP”); a copolymer or block polymer of PE and PP; and/or a polyurethane, an acrylic, a polyester, a polyamide, polyolefin, a polyimide, a vinyl acetate, an alkyd, an epoxy, a silane, or a siloxane; and mixtures thereof. This structure optionally includes at least one grommet disposed in the attachment edge or in one of the plurality of tabs.

According to yet a further embodiment of the disclosure, leaflet for a prosthetic replacement heart valve made of a polymer sheet material comprises: comprising a first major surface, a second major surface, an attachment edge and a free edge, said polymer sheet material composed of a polyethylene (“PE”), polypropylene (“PP”) or polytetrafluoroethylene (“PTFE”) and having indicia composed of a radiopaque material; and/or at least one grommet adjacent the attachment edge; and/or a plurality of tabs disposed adapted to form of commissures; and/or at least one grommet is disposed in each of the plurality of tabs; and/or at least one partial coating or partial layer disposed on a major surface at or adjacent the attachment edge; and/or at least one partial coating or partial layer disposed on a major surface at or adjacent the free edge.

According to yet a further embodiment of the disclosure, an expandable heart valve is contemplated including a cuff and/or leaflet made of a polymer sheet material comprising a first major surface, a second major surface, an attachment edge and a free edge, said polymer sheet material composed of a polyethylene (“PE”), polypropylene (“PP”) or polytetrafluoroethylene (“PTFE”) and having indicia composed of a radiopaque material; and/or at least one grommet adjacent the attachment edge; and/or a plurality of tabs disposed adapted to form of commissures; and/or at least one grommet is disposed in each of the plurality of tabs; and/or at least one partial coating or partial layer disposed on a major surface at or adjacent the attachment edge; and/or at least one partial coating or partial layer disposed on a major surface at or adjacent the free edge.

According to a further aspect of the disclosure; a prosthetic valve assembly comprises: a cuff; and three prosthetic leaflets sutured or laced to the cuff, at least one of the cuff and the leaflets composed of a polymer sheet material comprising a first major surface, a second major surface, an attachment edge, a free edge and a plurality of tabs and made of a polymer sheet material composed of a polyethylene (“PE”), polypropylene (“PP”), or polytetrafluoroethylene (“PTFE”).

According to an aspect of the disclosure, a process for assembling a medical device comprises: lacing a fiber through a grommet provided in a leaflet composed of a polymer sheet material of the disclosure; and attaching the leaflet to a stent, superstructure, support or a second leaflet or a cuff composed of a polymer sheet material of the disclosure using the fiber.

According to another embodiment of the disclosure, a method for forming a leaflet for use in a heart valve, includes: using a single sheet of material or layering a first sheet material on an additional sheet material; and repeating the layering step until the leaflet has a pre-desired thickness; and attaching it to a stent.

According to another embodiment of the disclosure, a method for forming a leaflet for use in a prosthetic heart valve, includes: folding a first portion of a sheet of material over a second portion of the sheet of material; and repeating the folding step until the leaflet has a selected thickness; and attaching it to a stent.

According to an additional embodiment of the disclosure, a leaflet for use in a prosthetic heart valve includes a pleat in a sewing region of the leaflet; the pleat capable of unfolding when a load is exerted on the leaflet during loading and reforms during valve opening; and/or the pleat is formed by folding a first portion of a sheet over a second portion of the polyurethane sheet.

According to one embodiment of the disclosure, a cuff and/or leaflet is provided for a replacement heart valve, where the cuff and/or leaflet is composed of a polymer sheet material. The polymer sheet material has at least one of the following properties: an ultimate tensile strength of from about 1 to about 500 MPa, a tear strength of from about 5 to about100 lbF, a permeability of from about lto about 2,000 mL/cm²/min, a suture retention of from about 10 to about 100 N, a stiffness/flexural rigidity of from about 0.001 to about 8 cm, and a stretch of from about 1 to about 400%. The leaflet has a thickness of from about 5 μm to about 500 μm and the cuff has a thickness of from about 1μm to about 300 tim. Another aspect of this embodiment is a valve assembly made with this cuff and/or leaflet and in still another aspect there is provided a surgical or expandable replacement heart valve including this valve assembly.

In another embodiment of the disclosure, the cuff and/or leaflet is composed of a polymer sheet material having at least one of the following properties: an ultimate tensile strength of from 25-250 MPa, a tear strength of 10-40 lbF, a permeability of 10-1,200 mL/cm²/min, a suture retention of 30-70 N, a stiffness/flexural rigidity of 0.001-4 cm and a stretch of 3-50%. The leaflet has a thickness of from about 50 μm to about 350 μm and the cuff has a thickness of from about 5 μm to about 200 tim. Another aspect of this embodiment is a valve assembly made with this cuff and/or leaflet and in still another aspect there is provided a surgical or expandable replacement heart valve including this valve assembly.

In a further embodiment of the disclosure, a replacement heart valve comprises: a self-expandable or balloon-expandable stent, and a valve assembly sutured to the stent. The valve assembly comprises at least one cuff attached to a luminal and/or an abluminal surface of the stent and two to three leaflets attached to the cuff and/or the luminal surface of the stent. The cuff(s) and leaflets are composed of polymer sheet materials composed of one or more layers of ultra-high molecular weight polyethylene or polytetrafluoroethylene which exhibit one of the following properties: an ultimate tensile strength of from about 25 to about 250 MPa; a tear strength of from about 10 to about40 lbF; a permeability of from about 10 to about 1,200 mL/cm²/min; a suture retention of from about 30 to about 70 N; a stiffness/flexural rigidity of from about 0.001 to about 4 cm or a stretch of from about 3 to about 50%. The leaflets have a thickness of from about 50 μm to about 350 μm and the cuff has a thickness of from about 5μm to about 200 tim.

In yet another embodiment of the disclosure, one of the leaflets and/or the cuffs comprises at least one polymer layer and attached thereto a second layer that: adjusts surface roughness; alters leaflet strength; provides added abrasion resistance; provides improved lubricity; adds weight to bias the leaflet to close; provides additional rigidity; promotes folding; alters cell adhesion; controls cellular ingrowth; or improves resistance to thrombosis, when compared to the cuff or leaflet without that layer; or includes a therapeutic agent when compared to the cuff or leaflet without that layer. In one aspect, this second layer is a partial layer disposed at or adjacent at least the free edge of the leaflet.

In another embodiment of the disclosure, a replacement heart valve comprises: a self-expandable or balloon-expandable stent, and a valve assembly sutured to the stent. The valve assembly comprises a cuff attached to a luminal and/or an abluminal surface of the stent, and a plurality of leaflets attached to the cuff and/or the luminal surface of the stent. The cuff and leaflets are composed of polymer sheet materials composed of one or more layers of ultra-high molecular weight polyethylene or polytetrafluoroethylene. At least one layer of the polymer sheet material exhibits one of the following properties: an ultimate tensile strength of from about 25 to about 250 MPa; a tear strength of from about 10 to about 40 lbF; a permeability of from about 10 to about 1,200 mL/cm²/min; a suture retention of from about 30 to about 70 N; a stiffness/flexural rigidity of from about 0.001 to about 4 cm or a stretch of from about 3 to about 50%. The leaflets have a thickness of from about 50 μm to about 350 μm and the cuff has a thickness of from about 5 μm to about 200 μm. One or more of the leaflets or the cuff further comprises a wire, stitch, a suture line or grommet. In the case of a stitch or a suture line, they are not provided to substantially attach the cuff or leaflet to another structure of the replacement heart valve.

And in yet another embodiment of the disclosure, there is provided a replacement heart valve as described above in the foregoing paragraphs which is specifically designed to replace or repair a native aortic or native mitral valve. In one embodiment of such a prosthetic mitral valve, the prosthetic mitral valve comprises a self-expandable or balloon-expandable stent that includes a first inner stent having a generally cylindrical shape and a second outer stent attached to the first inner stent. The second outer stent is disposed generally surrounding the first inner stent. When implanted into a native mitral valve annulus, the second outer stent engages the native valve annulus and at least partially mechanically isolates the first inner stent from being deformed by the anatomy of the native valve annulus or calcification of the native valve. Thus, the first inner stent retains its generally cylindrical shape. Moreover, the prosthetic mitral valve includes a valve assembly that is substantially only attached to the first inner stent. The valve assembly comprises at least one cuff attached to a surface of the stent and two or three leaflets attached to the cuff and/or the luminal surface of the first inner stent. The cuff(s) and/or leaflets are composed of polymer sheet materials composed of one or more layers of ultra-high molecular weight polyethylene or polytetrafluoroethylene. At least one layer of the polymer sheet material exhibits one of the following properties: an ultimate tensile strength of from about 25 to about 250 MPa; a tear strength of from about 10 to about 40 lbF; a permeability of from about 10 to about 1,200 mL/cm2/min: a suture retention of from about 30 to about 70 N; a stiffness/flexural rigidity of from about 0.001 to about 4 cm or a stretch of from about 3 to about 50%. The leaflets have a thickness of from about 50 μm to about 350 μm and the cuff has a thickness of from about 5 μm to about 200 μm. And is some additional aspect of this embodiment, the at least one of the leaflets or the cuff further comprises a wire, stitch, a suture line or grommet. In the case of a stitch or a suture line, they are not provided to substantially attach the cuff or leaflet to another structure of the replacement heart valve.

Although the present disclosure has been made with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims. For example, features of one embodiment described above may be combined with features of other embodiments described above.

1. A replacement heart valve comprising:
a self-expandable or balloon-expandable stent; and
a valve assembly sutured to the stent, the valve assembly comprising a cuff and a prosthetic leaflet composed of a polymer sheet material, the polymer sheet material having at least one property selected from the group consisting of (i) an ultimate tensile strength between 25 MPa and 250 MPa; (ii) a tear strength of between 10 lbF and 40 lbF; (iii) a permeability of between 10 mL/cm²/min and 1,200 mL/cm²/min; (iv) a suture retention of between 30 N and 70 N; (v) a stiffness/flexural rigidity of between 0.001 cm and 4 cm; and (vi) a stretch of between 3% and 50%,
wherein the prosthetic leaflet has a thickness of between about 5 μm and about 500 μm and the cuff has a thickness of between about 1 μm and about 300 μm.

2. The replacement heart valve of claim 1, wherein the prosthetic leaflet and the cuff each exhibit at least two properties selected from the group consisting of (i) an ultimate tensile strength of between 25 MPa and 250 MPa; (ii) a tear strength of between 10 lbF and 40 lbF; (iii) a permeability of between 10 mL/cm²/min and 1,200 mL/cm²/min; (iv) a suture retention of between 30 N and 70 N; (v) a stiffness/flexural rigidity of between 0.001 cm and 4 cm; and (vi) a stretch of between 3% and 50%.

3. The replacement heart valve of claim 1, wherein the replacement heart valve is configured to replace or repair a native aortic valve.

4. The replacement heart valve of claim 1, wherein the replacement heart valve is configured to replace or repair a native mitral valve.

5. The replacement heart valve of claim 4, wherein the stent includes a first inner stent having a generally cylindrical shape and a second outer stent generally surrounding the first inner stent, the second outer stent being attached to the first inner stent so that when the replacement heart valve is implanted into a native mitral valve annulus, the second outer stent engages the native mitral valve annulus and at least partially mechanically isolates the first inner stent from being deformed by the native mitral valve annulus or calcification of the native mitral valve such that the first inner stent retains the generally cylindrical shape, the valve assembly being substantially only attached to the first inner stent.

6. The replacement heart valve of claim 1, wherein the cuff is disposed on a luminal surface of the stent.

7. The replacement heart valve of claim 1, wherein the cuff is disposed on an abluminal surface of the stent.

8. The replacement heart valve of claim 1, wherein the cuff is disposed on an abluminal surface of the stent and includes structure to reduce paravalvular leaks around an outside of the stent.

9. The replacement heart valve of claim 8, further comprising a second cuff portion disposed on a luminal surface of the stent.

10. The replacement heart valve of claim 1, wherein the polymer sheet material is composed of a single polymer layer.

11. The replacement heart valve of claim 1, wherein the polymer sheet material is composed of a plurality of polymer layers.

12. The replacement heart valve of claim 1, wherein the polymer sheet material further comprises a partial layer at or adjacent a free edge of the prosthetic leaflet.

13. The replacement heart valve of claim 1, wherein the polymer sheet material further comprises a partial layer at or adjacent an attachment edge of the prosthetic leaflet.

14. The replacement heart valve of claim 13, wherein the polymer sheet material further comprises a second partial layer at or adjacent a free edge of the prosthetic leaflet.

15. The replacement heart valve of claim 1, further comprising, at or adjacent an attachment edge of the prosthetic leaflet, a wire, a suture line that does not attach the prosthetic leaflet to another structure, or a grommet.

16. The replacement heart valve of claim 1, further comprising, at or adjacent a free edge of the prosthetic leaflet, a wire, a suture line that does not attach the prosthetic leaflet to another structure, or a grommet.

17. The replacement heart valve of claim 1, further comprising at least one rib attached to the prosthetic leaflet.

18. The replacement heart valve of claim 1, wherein at least one of the cuff or the prosthetic leaflet is produced by three dimensional printing.

19. The replacement heart valve of claim 1, wherein the polymer sheet material is a first polymer sheet material, and the cuff is composed of a second polymer sheet material, at least one of the first polymer sheet material and the second polymer sheet material comprising a plurality of laminated layers or partial layers.

20. The replacement heart valve of claim 1, wherein the polymer sheet material is a first polymer sheet material, and the cuff is composed of a second polymer sheet material, at least one of the first polymer sheet material and the second polymer sheet material comprising a plurality of layers or partial layers and being contoured.

21. A replacement heart valve comprising:
a self-expandable or balloon-expandable stent; and
a valve assembly sutured to the stent, the valve assembly including at least one cuff attached to a luminal surface of the stent and an abluminal surface of the stent, and a plurality of prosthetic leaflets attached to the at least one cuff, the plurality of prosthetic leaflets being positioned within the stent,
wherein the at least one cuff is composed of a first polymer sheet material of ultra-high molecular weight polyethylene or polytetrafluoroethylene, and the plurality of prosthetic leaflets are each composed of a second polymer sheet material of ultra-high molecular weight polyethylene or polytetrafluoroethylene, the first and second polymer sheet materials each having at least two properties selected from the group consisting of: (i) an ultimate tensile strength of between 25 MPa and 250 MPa; (ii) a tear strength of between 10 lbF and 40 lbF; (iii) a permeability of between 10 mL/cm²/min and 1,200 mL/cm²/min; (iv) a suture retention of between 30 N and 70 N; (v) a stiffness/flexural rigidity of between 0.001 cm and 4 cm; and (vi) a stretch of between 3% and 50%,
wherein the plurality of prosthetic leaflets each have a thickness of between about 50 μm and about 350 tim, and the at least one cuff has a thickness of between about 5μm and about 200 tim.

22. The replacement heart valve of claim 21 wherein the second polymer sheet material further comprises:
an additional layer that, when compared to the prosthetic leaflet without the additional layer, (i) adjusts surface roughness; (ii) alters prosthetic leaflet strength; (iii) provides added abrasion resistance; (iv) provides improved lubricity; (v) adds weight to bias the prosthetic leaflet to close; (vi) provides additional rigidity; (vii) promotes folding; (viii) alters cell adhesion; (ix) controls cellular ingrowth; (x) improves resistance to thrombosis; or (xi) includes a therapeutic agent.

23. The replacement heart valve of claim 22, wherein the additional layer is a partial layer disposed at or adjacent a free edge of the prosthetic leaflet.

24. The replacement heart valve of claim 21, wherein the second sheet material further comprises a partial layer at or adjacent an attachment edge of the prosthetic leaflet.

25. The replacement heart valve of claim 21, further comprising (i) a wire, (ii) a suture line that does not attach the at least one cuff or the plurality of prosthetic leaflets to another structure, or (iii) a grommet, attached to the at least one cuff or the plurality of prosthetic leaflets.
